SEQUENCE LISTING (12 sequences)

SEQ ID NO: 1 - protein, 12 aa, Arabidopsis thaliana
MASSMLSSATMV

SEQ ID NO: 2 - protein, 12 aa, synthetic construct
KHKHKHKHKHKH

SEQ ID NO: 3 - protein, 6 aa, synthetic construct
GGGGGG

SEQ ID NO: 4 - protein, 30 aa, synthetic construct
MASSMLSSATMVGGGGGGKHKHKHKHKHKH

SEQ ID NO: 5 - protein, 15 aa, synthetic construct
MASSMLSSATMVGGC

SEQ ID NO: 6 - DNA, 30 bp, synthetic construct
  source 1..30: mol_type = other DNA
  modified_base 1: mod_base = OTHER; note = Cyanine-3 modified nucleotide
gtgtgtgtgtgtgtgtgtgtgtgtgtgtgt

SEQ ID NO: 7 - DNA, 24 bp, synthetic construct
  source 1..24: mol_type = other DNA
cacaatgtttggcgggattggtga

SEQ ID NO: 8 - DNA, 24 bp, synthetic construct
  source 1..24: mol_type = other DNA
tgtacttcctttccggtggagcaa

SEQ ID NO: 9 - DNA, 22 bp, synthetic construct
  source 1..22: mol_type = other DNA
ctgtcagtggagagggtgaagg

SEQ ID NO: 10 - DNA, 22 bp, synthetic construct
  source 1..22: mol_type = other DNA
caagtgttggccaaggaacagg

SEQ ID NO: 11 - protein, 20 aa, synthetic construct
  VARIANT 1..20: note = This sequence may encompass 2-10 KH repeating units
KHKHKHKHKHKHKHKHKHKH

SEQ ID NO: 12 - protein, 10 aa, synthetic construct
  VARIANT 1..10: note = This sequence may encompass 1-10 residues
GGGGGGGGGG

(19) United States
(12) Patent Application Publication
(10) Pub. No.: US 2023/0313217 A1
(43) Pub. Date: Oct. 5, 2023

(54) CHLOROPLAST TARGETING PEPTIDES AND CONJUGATES AND COMPOSITIONS THEREOF

(71) Applicant: The Regents of the University of California, Oakland, CA (US)

(72) Inventors: Juan Pablo Giraldo Gomez, Riverside, CA (US); Israel Santana, Riverside, CA (US); Gregory Newkirk, Riverside, CA (US)

(21) Appl. No.: 18/070,288

(22) Filed: Nov. 28, 2022

Related U.S. Application Data
(60) Provisional application No. 63/283,849, filed on Nov. 29, 2021.

(51) Int. Cl. C12N 15/82; C07K 14/415; A01N 63/60; A01N 25/08

(52) U.S. Cl. CPC C12N 15/8269; C07K 14/415; A01N 63/60; A01N 25/08

(57) ABSTRACT

Certain embodiments provide a polypeptide comprising a chloroplast-targeting amino acid sequence linked to an amino acid sequence that is capable of electrostatically binding to a nucleic acid molecule, as well as conjugates comprising such polypeptides and methods of use thereof.

### CROSS-REFERENCE TO RELATED APPLICATION

This application claims priority to U.S. provisional Application No. 63/283,849 filed 29 Nov. 2021, the contents of which are hereby incorporated by reference in their entirety.

### GOVERNMENT FUNDING

This invention was made with government support under 1911763 awarded by the National Science Foundation and 2019-67013-29104 awarded by National Institute of Food and Agriculture, USDA. The government has certain rights in the invention.

### SEQUENCE LISTING

The instant application contains a Sequence Listing which has been submitted electronically in XML format and is hereby incorporated by reference in its entirety. Said XML copy, created on Jun. 13, 2023, is named 12111_050US1_SL.xml and is 12,948 bytes in size.

### BACKGROUND OF THE INVENTION

Chloroplasts are photosynthetic, semi-autonomous organelles that are essential for fixing carbon in plants. In addition, chloroplasts act as signaling organelles and play key roles in the synthesis of metabolites. These photosynthetic plastids, which have a prokaryotic-like genome, are excellent targets for genetic engineering tools due to the ability to isolate genetic markers in parental lines, minimize outcrossing of transgenes to other crops, use multiple genes encoded in one plasmid, and the lack of silencing mechanisms. However, compositions and methods for targeted delivery of nucleic acids to the chloroplast are limited.

There is a need for new methods and compositions for chloroplast-targeted nucleic acid delivery to plants.

### SUMMARY OF THE INVENTION

Certain embodiments provide a polypeptide comprising a chloroplast-targeting amino acid sequence linked to an amino acid sequence that is capable of electrostatically binding to a nucleic acid molecule.

Certain embodiments provide a polynucleotide comprising a nucleic acid sequence encoding a polypeptide as described herein.

Certain embodiments provide a conjugate comprising one or more polypeptides as described herein and a nucleic acid, wherein the one or more polypeptides are electrostatically bound to the nucleic acid.

Certain embodiments also provide a conjugate comprising one or more polypeptides as described herein, one or more nucleic acids, and a functionalized nanoparticle, wherein the one or more nucleic acids are electrostatically bound to the functionalized nanoparticle, wherein the one or more polypeptides are electrostatically bound to the one or more nucleic acids, and wherein the functionalized nanoparticle is selected from the group consisting of a functionalized quantum dot, carbon dot, carbon nanotube, silica nanoparticle, and metal or metal oxide nanoparticle.

Certain embodiments provide a composition comprising a polypeptide as described herein or a conjugate as described herein and a carrier.

Certain embodiments provide a method of delivering one or more nucleic acids to a chloroplast of a plant, the method comprising introducing to the plant a conjugate as described herein.

Certain embodiments provide a method of expressing a target protein in the chloroplast of a plant, the method comprising introducing to the plant (e.g., a leaf) a conjugate as described herein, wherein the conjugate comprises one or more nucleic acids encoding the target protein.

Certain embodiments provide a method of treating a disease in a plant, the method comprising introducing to the plant (e.g., a leaf) an effective amount of a conjugate as described herein, wherein the conjugate comprises one or more nucleic acids effective to treat the disease.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Targeted carbon nanostructures for chloroplast bioengineering and their impact on the plant cell and molecular biology. Nanomaterials were synthesized for chloroplast targeted chemical delivery (CDs) and gene delivery (SWCNTs). The carbon nanostructures were functionalized with a guiding peptide that selectively binds to protein translocon outer channels (TOC) on the chloroplast membrane. Nanomaterials have been proposed to spontaneously enter plant cells and organelles by disrupting lipid bilayers. The impact of targeted carbon nanostructures on leaf cell and molecular biology was assessed by studying the effects on plant cell and chloroplast membrane integrity, the damage to whole plant cell and isolated chloroplast DNA, the generation of ROS, and photosynthesis. Created with BioRender.com.

FIGS. 2A-2C. Characterization of carbon nanostructures for chemical and gene delivery to chloroplasts. (FIG. 2A) ζ potential and (FIG. 2B, FIG. 2C) hydrodynamic diameter of targeted and nontargeted carbon nanostructures. CDs with β-cyclodextrin molecular baskets (β-CDs) and chloroplast targeting peptides (TP-β-CDs). PEI-SWCNTs coated with pATV1 plasmid DNA (pATV1-SWCNT), CY3-ds(GT)₁₅-SWCNT and nanostructures with chloroplast targeting peptide (TP-pATV1-SWCNT, TP-CY3-ds(GT)₁₅-SWCNT).

FIGS. 3A-3C. Targeted delivery of nanomaterials to chloroplasts in plant cells. (FIG. 3A) Confocal microscopy images of CDs with β cyclodextrin molecular baskets (β-CDs) and chloroplast targeting peptides (TP-β-CDs) and (FIG. 3B) single walled carbon nanotubes coated with CY3 with double-stranded DNA (CY3-ds(GT)₁₅-SWCNT) and chloroplast targeting peptides (TP-CY3-ds(GT)₁₅-SWCNT). Confocal images were collected in the same focal plane about 50 μm from the leaf abaxial epidermis. Scale bar 50 μm. (FIG. 3C) Colocalization analysis of nanostructures indicated a significantly higher percentage of chloroplasts with targeted nanomaterials compared to controls without TPs. Statistical analysis using a one-way ANOVA and post hoc Tukey's test, n=7-12, ****p<0.0001. FIG. 3B discloses SEQ ID NO: 6.

FIGS. 4A-4C. Chemical cargo delivery by targeted CD nanostructures. (FIG. 4A) CDs are functionalized with a β-cyclodextrin molecular baskets for chemical cargo delivery (e.g., fluorescent dye) and with chloroplast guiding peptide for targeted delivery enabled by biorecognition. (FIG. 4B) Confocal microscopy images of fluorescein (FDA) in leaf mesophyll cells indicate a higher degree of colocalization with chloroplasts for targeted TP-β-CD-FDA compared to nontargeted β-CD-FDA, FDA alone, or a mixture of CDs and FDA. Merged confocal images show 2D Z-projections to visualize the distribution of fluorescent cargoes in leaf mesophyll cells and chloroplasts. Confocal images were collected in the same focal plane about 50 μm from the leaf abaxial epidermis. Scale bar 50 μm. (FIG. 4C) Quantitative colocalization analysis of the percentage of chloroplasts containing FDA. Statistical analysis was performed using a one-way ANOVA and post hoc Tukey's test, n=7, ***p<0.0002, ****p<0.0001.

FIGS. 5A-5F. Plasmid DNA delivery to chloroplasts by targeted SWCNTs. (FIG. 5A) SWCNTs coated with PEI are electrostatically bound to plasmid DNA and chloroplast targeting peptides for chloroplast genetic engineering. The pATV1 plasmid encodes for a GFP gene. Gene map of pATV1 vector contains plastid operon prrn16 and dicistronic open reading frames for GFP and spectinomycin resistance genes. The chloroplast targeting peptide was rationally designed to include amino acid sequences for biorecognition, increased flexibility and stability, and an electrostatic tail to bind to DNA. The chloroplast targeting peptide contains a chloroplast biorecognition motif (Red) from the rubisco small subunit 1 A transit peptide and a middle section (Green) with a flexible linker of six glycine residues (SEQ ID NO: 3) for increased stability and interaction between domains. Lastly, the terminal end of the fusion peptide contains a lysine histidine (KH)₆ polypeptide tail (SEQ ID NO: 2) (Blue) for electrostatic interactions with the DNA backbone grafted onto the PEI-SWCNT. (FIG. 5B) Confocal microscopy images of Arabidopsis thaliana leaf mesophyll cells after 7 days of exposure to SWCNTs coated in pATV1 plasmid with targeting peptides (TP-pATV1-SWCNTs) and without the guiding peptides (pATV1-SWCNTs). Scale bar 50 μm. (FIG. 5C) 3D image of GFP in leaf mesophyll cells after treatment with TP-pATV1-SWCNT. (FIG. 5D) Quantitative colocalization analysis of chloroplasts with GFP fluorescence. Comparisons were performed by independent samples t-test (two-tailed, ****p=<0.0001, n=5-7). (FIG. 5E) RT-qPCR gene expression analysis of GFP in plants treated with targeted TP-pATV1-SWCNTs and nontargeted pATV1-SWCNTs. Data are the means ±SD (n=5-7). (FIG. 5F) ELISA quantification of GFP in total soluble proteins of extracts from leaves treated with targeted TP-pATV1-SWCNTs and nontargeted pATV1-SWCNT. Data are means ±SD (n=3). Statistical analysis was performed with a two-way ANOVA. No significant differences (ns). FIG. 5A discloses SEQ ID NO: 4.

FIGS. 6A-6B. Biocompatibility of targeted nanomaterials in plant cells. Percentage of dead cells in Arabidopsis leaf mesophyll tissue exposed to increasing concentrations of targeted nanomaterials. (FIG. 6A) TP-β-CDs or (FIG. 6B) TP-pATV1-SWCNTs. The percentage of dead cells was determined using PI, a fluorescent dye that stains the nuclei of dead cells. Statistical analysis using one-way ANOVA and post hoc Dunnett's test, n=7-12, *p<0.032, **p=<0.0021. In FIG. 6A, for each grouping the following are shown from left to right: Buffer, 20 mg/L, 100 mg/L, and 500 mg/L.

FIGS. 7A-7D. Impact of targeted nanomaterials on plant cell and chloroplast membrane integrity. (FIG. 7A) Confocal microscopy images of leaf mesophyll cell dead nuclei stained with PI. The PI a nonpermeable dye that can only cross plant cell membranes when they are damaged. White arrows point to selected stained nuclei. Scale bar 50 μm. (FIG. 7B) Quantitative analysis of the percentage of intact plant cells based on confocal microscopy imaging. Statistical analysis using one way ANOVA and post hoc Dunnett's test. No significant differences (ns), n=5-7. (FIG. 7C) Bright-field microscopy images of isolated chloroplasts for intact chloroplast analysis. Intact chloroplasts exhibited a highly reflective and continuous outer membrane (white arrows) under DIC optical imaging. In contrast, the damaged chloroplasts showed broken outer membranes giving a granular appearance (yellow arrows with asterisk). Scale bar 10 μm. (FIG. 7D) Quantitative analysis of the percentage of intact plant chloroplast membranes based on DIC optical imaging. Statistical analysis using one-way ANOVA and post hoc Tukey test. *p<0.032, n=5-7. In FIGS. 7B and 7D, for each grouping the following are shown from left to right: Buffer, TP-β-CD and TP-pATV1-SWCNT.

FIGS. 8A-8C. Oxidative stress in leaf mesophyll cells exposed to targeted nanomaterials. (FIG. 8A) Hydrogen peroxide (H₂O₂) content in Arabidopsis leaves treated with targeted nanostructures 1 and 5 days after exposure. Statistical analysis using two-way ANOVA and post hoc Tukey's test n=9, *p=<0.05, **p<0.0021, ****p<0.0001, n=3. Quantitative assay of DNA damage caused by oxidative stress in (FIG. 8B) whole leaf cells and (FIG. 8C) isolated chloroplasts. The biomarker 8-OHdG was measured by ELISA, and the relative percentages of the 8-OHdG levels were compared to controls (buffer without nanoparticles) at 1 and 5 days after treatment with targeted nanostructures. Statistical analysis performed by one-way ANOVA and post hoc Tukey test **p<0.0021, ***p<0.0002, ****p<0001, n=6. For each grouping the following are shown from left to right: Buffer, TP-β-CD and TP-pATV1-SWCNT.

FIGS. 9A-9F. Effect of targeted carbon nanostructures on plant photosynthesis. Comparison of chlorophyll content index (SPAD) in Arabidopsis leaves treated with targeted nanomaterials at (FIG. 9A) day 1 and (FIG. 9B) day 5 after exposure. Statistical analysis was performed by one-way ANOVA and post hoc Tukey's test. * p<0.02, **p<0.0021, ***p<0.0002, ****p<0.0001, n=6. Leaf carbon assimilation rates at varied photosynthetic active radiation (PAR) levels of Arabidopsis leaves at (FIG. 9C) day 1 and (FIG. 9D) day 5 after exposure to targeted nanomaterials. Statistical analysis was performed by two-way ANOVA and post hoc Dunnett's test. *p<0.02, **p<0.0021, ***p<0.0002, ****p<0.0001, n=7-10. Quantum yield of PSII and maximum efficiency of photosystem II (Fᵥ/Fₘ) at (FIG. 9E) day 1 and (FIG. 9F) day 5 after exposure to targeted nanomaterials. In FIGS. 9A-9D, 9E inset graph, and 9F, for each grouping the following are shown from left to right: Buffer, TP-β-CD, and TP-pATV1-SWCNT. In the main graph of FIG. 9E, the following are shown from left to right: TP-β-CD, TP-pATV1-SWCNT and Buffer.

FIGS. 10A-10D. Characterization of targeted carbon nanostructures for chemical and gene delivery to chloroplasts. FIG. 10A, FIG. 10B, UV vis absorbance of targeted and non-targeted carbon nanostructures. Carbon dots (CD) with β-cyclodextrin molecular baskets (β-CD) and chloroplast targeting peptides (TP-β-CD). PEI coated single walled carbon nanotubes (PEI-SWCNT) coated with pATV1 plasmid DNA and chloroplast targeting peptide (TP-pATV1-SWCNT). Fluorescence emission spectra of FIG. 10C, TP-β-CD (405 nm excitation) and FIG. 10D, TP-CY3-ds(GT)₁₅-SWCNT (488 nm excitation) do not overlap with chloroplast autofluorescence.

FIG. 11. FTIR spectra of step-by-step synthesis of targeted carbon dot nanostructures. The bottom panel shows spectra of core carbon dots, the middle panel represents spectra of β-CD, and the upper panel indicates the spectra for TP-β-CD with characteristic bonds.

FIGS. 12A-12B. Control confocal microscopy images of Arabidopsis leaf mesophyll cells without nanoparticles. Leaves treated with 10 mM TES buffer showed no signal in the emission range used for detection of FIG. 12A, carbon dots (CD) and FIG. 12B, CY3-ds(GT)₁₅-SWCNT. Scale bar 50 μm. FIG. 12B discloses SEQ ID NO: 6.

FIGS. 13A-13B. Imaging and fluorescence emission of TP-β-CD cargo delivery experiments. FIG. 13A, Confocal microscopy images of plant leaves exposed to TP-β-CDs with and without loading of fluorescein (FDA) cargo. There is no fluorescence emission background of the TP-β-CD in the FDA channel at 488 nm laser excitation. FIG. 13B, the fluorescence emission spectra of TP-β-CDs, FDA dye and chloroplast autofluorescence under a laser excitation at 488 nm shows a more intense fluorescence emission from FDA than TP-β-CDs, and a distinct emission from chloroplast fluorescence. Confocal microscopy settings were selected to remove fluorescence background from TP-β-CD in FDA channel as shown in the control without FDA (FIG. 13A, first row). Scale bar 50 μm.

FIG. 14. Control confocal microscopy images of Arabidopsis thaliana leaf mesophyll cells exposed to PEI-SWCNT. No fluorescence signal in GFP emission range was detected in leaves after 7 days of exposure to PEI-SWCNT. Scale bar 50 μm.

FIGS. 15A-15B. Leaf chlorophyll content index (CCI). FIG. 15A, Leaf CCI levels were monitored to up to 7 days when a significant difference in chlorophyll content was detected due to leaf senescence. Statistical analysis was performed with a one-way ANOVA based post-hoc Tukey's test. n=7-12, *p<0.032. FIG. 15B, No significant differences in leaf CCI were observed between non-treated controls plants, plants infiltrated with buffer, or plants exposed to targeted nanostructures after 3 h. No significant (ns), n=7-12.

FIG. 16. Images of 3-week-old Arabidopsis thaliana plants exposed to targeted nanomaterials after one and five days. Scale bar 2.5 cm.

FIG. 17. Quantification of formulation amount remaining on Arabidopsis leaves after foliar spraying. Arabidopsis plants on petri dishes were weighed before & after being foliar sprayed with 900 μL of nanomaterial formulation in Silwet L-77. Out of 900 μL of formulation, 581.9 μL±0.05 reach the plant and the petri dish, and 307.8±0.05 μL remain on the plant leaves. These results indicate that 34.2% of the total volume sprayed remains on the plant leaves. Calculation of volumes was conducted as follows: 1. Plant and petri dish=Weight (C−B)/density of the formulation*. 2. Plant leaves=Weight (C−D−A)/density of the formulation*. * Density of buffer in Silwet L-77=1.0158 g/mL

FIGS. 18A-18B. Primer design and efficiency testing for expression analysis. FIG. 18A, Linear plasmid map of pATV1 expression vector. Lower boxed image shows a split enlarged version of the linear plasmid map. Expression analysis primers were designed with Primer3 version 4.1.0 with the pATV1 sequence from Lu and colleagues (Koressaar and Remm 2007; Yu et al. 2017). Five primer sets were ordered from Integrated DNA Technologies; the primer pair, AtGFP mEA F1=5′-ctgtcagtggagagggtgaagg-3′ (SEQ ID NO:9), AtGFP mEA R1=5′-caagtgttggccaaggaacagg-3′ (SEQ ID NO:10), produced a 99 bp amplicon, pictured here within Benchling. FIG. 18B, Primer validation experiment with serial dilutions of pATV1 plasmid and Arabidopsis thaliana cDNA were tested with triplicate RT-qPCR reactions, and the previously mentioned primer pair was calculated with a priming efficiency of E=1.332, where E=−1+10\hat{ }(−1/slope), and y=−2.7186x+8.7215; melt curve analysis confirmed a single amplicon was made (Pfaffl, et al., Nucleic Acids Res. 29, e45 (2001)). No-template controls Ct values matched the wildtype Arabidopsis cDNA, so off-target binding was deemed not relevant.

FIG. 19. Circular plasmid map of the pATV1 expression vector. Expression analysis primers were designed with Primer3 version 4.1.0 with the pATV1 sequence from Lu and colleagues (Koressaar, T., & Remm, M. (2007). Bioinformatics, 23(10), 1289-1291; Yu, et al., (2017). Plant Physiology, 175(1), 186-193). Five primer sets were ordered from Integrated DNA Technologies; the primer pair, AtGFP mEA F1=5′-ctgtcagtggagagggtgaagg-3′ (SEQ ID NO:9), AtGFP mEA R1=5′-caagtgttggccaaggaacagg-3′ (SEQ ID NO:10), produced a 99 bp amplicon, pictured here within Benchling.

FIG. 20. Quantitative RT-qPCR primer list.

FIG. 21. DNA Oligos of CY3-Tagged DNA sequence (CY3GTGTGTGTGTGTGTGTGTGTGTGTGTGTGT (SEQ ID NO:6)). Figure discloses SEQ ID NO: 6.

### DETAILED DESCRIPTION

### Chloroplast Targeting Polypeptides

Certain embodiments provide a polypeptide comprising a chloroplast-targeting amino acid sequence linked to an amino acid sequence that is capable of electrostatically binding to a nucleic acid molecule.

As used herein, the term “chloroplast-targeting amino acid sequence” refers to an amino acid sequence that facilitates translocation across chloroplast membranes. In certain embodiments, the chloroplast-targeting amino acid sequence is capable of being recognized by cytosolic machinery within a plant cell, which then facilitates the translocation. For example, in certain embodiments, the chloroplast-targeting amino acid sequence is capable of being recognized by a translocon supercomplex on the chloroplast outer membrane (TOC), such as TOC159. In certain embodiments, the chloroplast-targeting amino acid sequence is capable of being recognized by and binding to a chloroplast heat shock protein 70 (cpHsp70).

In certain embodiments, the chloroplast-targeting amino acid sequence is at least about 5, 10, 15, 20, 25, 30, 35, 40, 50, 60, 70, 80, 90, or 100 amino acids in length. In certain embodiments, the chloroplast-targeting amino acid sequence is between about 5 to 100 amino acids in length, or about 5 to 75 amino acids in length, or about 5 to 50 amino acids in length, or about 5 to 30 amino acids in length, or about 5 to 20 amino acids in length, or about 8 to 16 amino acids in length. In certain embodiments, the chloroplast-targeting amino acid sequence is about 8, 9, 10, 11, 12, 13, 14, 15, or 16 amino acids in length. In certain embodiments, the chloroplast-targeting amino acid sequence is about 12 amino acids in length.

In certain embodiments, the chloroplast-targeting amino acid sequence comprises an amino acid sequence of a ribulose bisphosphate carboxylase small chain 1A (RBCS1A) protein. In certain embodiments, the chloroplast-targeting amino acid sequence comprises an amino acid sequence of a dicot RBCS1A protein. An example of such a chloroplast-targeting amino acid sequence is MASSMLSSATMV (SEQ ID NO: 1), which is derived from the Arabidopsis thaliana RBCS1A (genbank: OAP15425) protein and highly conserved among many dicotyledon plants (see, also, Santana et al., Nat Commun 11, 2045 (2020), which is incorporated by reference herein in its entirety for all purposes).

Thus, in certain embodiments, the chloroplast-targeting amino acid sequence comprises an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:1. In certain embodiments, the chloroplast-targeting amino acid sequence comprises an amino acid sequence having at least about 75% sequence identity to SEQ ID NO:1. In certain embodiments, the chloroplast-targeting amino acid sequence comprises an amino acid sequence having at least about 80% sequence identity to SEQ ID NO:1. In certain embodiments, the chloroplast-targeting amino acid sequence comprises an amino acid sequence having at least about 90% sequence identity to SEQ ID NO:1. In certain embodiments, the chloroplast-targeting amino acid sequence comprises SEQ ID NO:1.

In certain embodiments, the chloroplast-targeting amino acid sequence consists of an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:1. In certain embodiments, the chloroplast-targeting amino acid sequence consists of an amino acid sequence having at least about 75% sequence identity to SEQ ID NO:1. In certain embodiments, the chloroplast-targeting amino acid sequence consists of an amino acid sequence having at least about 80% sequence identity to SEQ ID NO:1. In certain embodiments, the chloroplast-targeting amino acid sequence consists of amino acid sequence having at least about 90% sequence identity to SEQ ID NO: 1. In certain embodiments, the chloroplast-targeting amino acid sequence consists of SEQ ID NO:1.

As described herein, a polypeptide of the invention also comprises an amino acid sequence that is capable of electrostatically binding to a nucleic acid. For example, in certain embodiments, such an amino acid sequence comprises one or more positively charged groups capable of electrostatically binding to the negatively charged backbone of a nucleic acid molecule. In certain embodiments, the amino acid sequence comprises one or more (e.g., 1, 2, 3, 4, 5, 6, 7, 8, 9, 10, 11, 12 or more) amino acid residues having at least one protonated amine group. Thus, in certain embodiments, the amino acid sequence that is capable of electrostatically binding to a nucleic acid comprises one or more positively charged amino acids, such as lysine, histidine or arginine residues.

In certain embodiments, the amino acid sequence that is capable of electrostatically binding to a nucleic acid is a lysine-rich, histidine-rich and/or arginine-rich sequence. As used herein, an amino acid sequence that is “rich” in a particular amino acid or a combination of amino acids is a sequence having at least about 50% of the amino acids within the sequence being the particular amino acid or the specified combination of amino acids. In certain embodiments, at least about 50%, 60%, 70%, 80%, 90% or 100% of the amino acids in the sequence are the specified amino acid(s). In certain embodiments, the amino acid sequence that is capable of electrostatically binding to a nucleic acid is a lysine- and histidine-rich sequence. In certain embodiments, the amino acid sequence that is capable of electrostatically binding to a nucleic acid is a lysine- and histidine-rich sequence, wherein at least about 60%, 80%, or 100% of the amino acids in the sequence are lysine or histidine.

In certain embodiments, the amino acid sequence that is capable of electrostatically binding to a nucleic acid is at least about 5, 10, 15, 20, 25, 30, 35, 40 or 50 amino acids in length. In certain embodiments, the amino acid sequence that is capable of electrostatically binding to a nucleic acid is between about 5 to 50 amino acids in length, or about 5 to 30 amino acids in length, or about 5 to 20 amino acids in length, or about 8 to 16 amino acids in length. In certain embodiments, the amino acid sequence that is capable of electrostatically binding to a nucleic acid is about 8, 9, 10, 11, 12, 13, 14, 15, or 16 amino acids in length. In certain embodiments, the amino acid sequence that is capable of electrostatically binding a nucleic acid is about 12 amino acids in length.

In certain embodiments, the amino acid sequence that is capable of electrostatically binding to a nucleic acid comprises a (KH)ₙ sequence, wherein n is 2, 3, 4, 5, 6, 7, 8, 9, 10 or more (SEQ ID NO: 11). In certain embodiments, n is 4, 6, or 8. In certain embodiments, n is 6.

In certain embodiments, the amino acid sequence that is capable of electrostatically binding to a nucleic acid comprises an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to KHKHKHKHKHKH (SEQ ID NO:2). In certain embodiments, the amino acid sequence that is capable of electrostatically binding to a nucleic acid comprises an amino acid sequence having at least about 75% sequence identity to SEQ ID NO:2. In certain embodiments, the amino acid sequence that is capable of electrostatically binding to a nucleic acid comprises an amino acid sequence having at least about 80% sequence identity to SEQ ID NO:2. In certain embodiments, the amino acid sequence that is capable of electrostatically binding to a nucleic acid comprises an amino acid sequence having at least about 90% sequence identity to SEQ ID NO:2. In certain embodiments, the amino acid sequence that is capable of electrostatically binding to a nucleic acid comprises SEQ ID NO:2.

In certain embodiments, the amino acid sequence that is capable of electrostatically binding to a nucleic acid consists of an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:2. In certain embodiments, the amino acid sequence that is capable of electrostatically binding to a nucleic acid consists of an amino acid sequence having at least about 75% sequence identity to SEQ ID NO:2. In certain embodiments, the amino acid sequence that is capable of electrostatically binding to a nucleic acid consists of an amino acid sequence having at least about 80% sequence identity to SEQ ID NO:2. In certain embodiments, the amino acid sequence that is capable of electrostatically binding to a nucleic acid consists of amino acid sequence having at least about 90% sequence identity to SEQ ID NO:2. In certain embodiments, the amino acid sequence that is capable of electrostatically binding to a nucleic acid consists of SEQ ID NO:2.

As described herein, polypeptides of the invention comprise a chloroplast-targeting amino acid sequence that is linked to an amino acid sequence that is capable of electrostatically binding to a nucleic acid. The chloroplast-targeting amino acid sequence may be linked to the amino acid sequence that is capable of electrostatically binding to a nucleic acid through a peptide bond or through a polypeptide linker.

In certain embodiments, the two sequences are linked through a peptide bond.

In certain embodiments, the two sequences are linked by a polypeptide linker. In certain embodiments, the polypeptide linker is about 1 to about 20 amino acids in length, or about 1 to about 15 amino acids in length, or about 1 to about 10 amino acids in length (e.g., 1, 2, 3, 4, 5, 6, 7, 8, 9 or 10 amino acids in length).

In certain embodiments, the polypeptide linker is a flexible polypeptide linker. In certain embodiments, the polypeptide linker is a glycine-rich linker (e.g., at least about 60%, 80% or 100% of the amino acids in the sequence are glycine). In certain embodiments, the polypeptide linker comprises (G)ₙ sequence, wherein n is 1, 2, 3, 4, 5, 6, 7, 8, 9, 10 or more (SEQ ID NO: 12). In certain embodiments, n is 1 to 10. In certain embodiments, n is 6.

In certain embodiments, the polypeptide linker comprises an amino acid sequence having at least about 60% sequence identity to GGGGGG (SEQ ID NO:3). In certain embodiments, the polypeptide linker comprises an amino acid sequence having at least about 80% sequence identity to SEQ ID NO: 3. In certain embodiments the polypeptide linker comprises SEQ ID NO: 3. In certain embodiments, the polypeptide linker consists of SEQ ID NO: 3.

In certain embodiments, a polypeptide of the invention as described herein is at least about 10, 15, 20, 25, 30, 35, 40, 50, 60, 70, or 80 amino acids in length. In certain embodiments, a polypeptide as described herein is between about 10 to 80 amino acids in length, or about 10 to 60 amino acids in length, or about 10 to 40 amino acids in length, about 20 to 40 amino acids in length, or about 25 to 35 amino acids in length. In certain embodiments, a polypeptide as described herein is about 25, 26, 27, 28, 29, 30, 31, 32, 33, 34, or 35 amino acids in length. In certain embodiments, a polypeptide as described herein is about 30 amino acids in length.

In certain embodiments, a polypeptide as described herein comprises an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:4. In certain embodiments, a polypeptide as described herein comprises an amino acid sequence having at least about 80% sequence identity to MASSMLSSATMVGGGGGGKHKHKHKHKHKH (SEQ ID NO:4). In certain embodiments, a polypeptide as described herein comprises an amino acid sequence having at least about 85% sequence identity to SEQ ID NO:4. In certain embodiments, a polypeptide as described herein comprises an amino acid sequence having at least about 90% sequence identity to SEQ ID NO:4. In certain embodiments, a polypeptide as described herein comprises an amino acid sequence having at least about 95% sequence identity to SEQ ID NO:4. In certain embodiments, a polypeptide as described herein comprises SEQ ID NO:4.

In certain embodiments, a polypeptide as described herein consists of an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:4. In certain embodiments, a polypeptide as described herein consists of an amino acid sequence having at least about 80% sequence identity to SEQ ID NO:4. In certain embodiments, a polypeptide as described herein consists of an amino acid sequence having at least about 85% sequence identity to SEQ ID NO:4. In certain embodiments, a polypeptide as described herein consists of amino acid sequence having at least about 90% sequence identity to SEQ ID NO:4. In certain embodiments, a polypeptide as described herein consists of amino acid sequence having at least about 95% sequence identity to SEQ ID NO:4. In certain embodiments, a polypeptide as described herein consists of SEQ ID NO:4.

Certain embodiments of the invention provide a polypeptide as described herein.

In some embodiments, the polypeptides described herein are prepared using recombinant methods. Accordingly, certain embodiments provide polynucleotides (e.g., isolated polynucleotides) comprising a nucleic acid sequence encoding any of the polypeptides described herein. The polynucleotides may be single-stranded or double-stranded. In some embodiments, the polynucleotide is DNA. In some embodiments, the polynucleotide is cDNA. In some embodiments, the polynucleotide is RNA. In certain embodiments, the nucleic acid further comprises a promoter.

Certain embodiments of the invention provide an expression cassette comprising a nucleic acid sequence encoding a polypeptide of the invention and a promoter operably linked to the nucleic acid. In certain embodiments, the promoter is a regulatable promoter. In certain embodiments, the promoter is a constitutive promoter. In certain embodiments, the expression cassette further comprises an expression control sequence (e.g., an enhancer) operably linked to the nucleic acid sequence. Expression control sequences and techniques for operably linking sequences together are well known in the art.

Nucleic acids/expression cassettes encoding a polypeptide described herein can be engineered into a vector using standard ligation techniques, such as those described in Sambrook and Russell, Molecular Cloning: A Laboratory Manual, Cold Spring Harbor Laboratory Press Cold Spring Harbor, NY (2001). Accordingly, certain embodiments provide a vector comprising an expression cassette comprising a promoter operably linked to a nucleic acid sequence encoding a polypeptide as described herein. Non-limiting examples of vectors include plasmids and viral expression systems.

Certain embodiments of the invention provide a cell comprising a polypeptide described herein, a nucleic acid encoding a polypeptide as described herein, an expression cassette comprising such a nucleic acid, or a vector comprising a nucleic acid encoding a polypeptide of the invention or an expression cassette comprising a nucleic acid encoding a polypeptide of the invention. In certain embodiments, the cell is a plant cell. In certain embodiments, the cell is a bacterial cell.

### Certain Conjugates

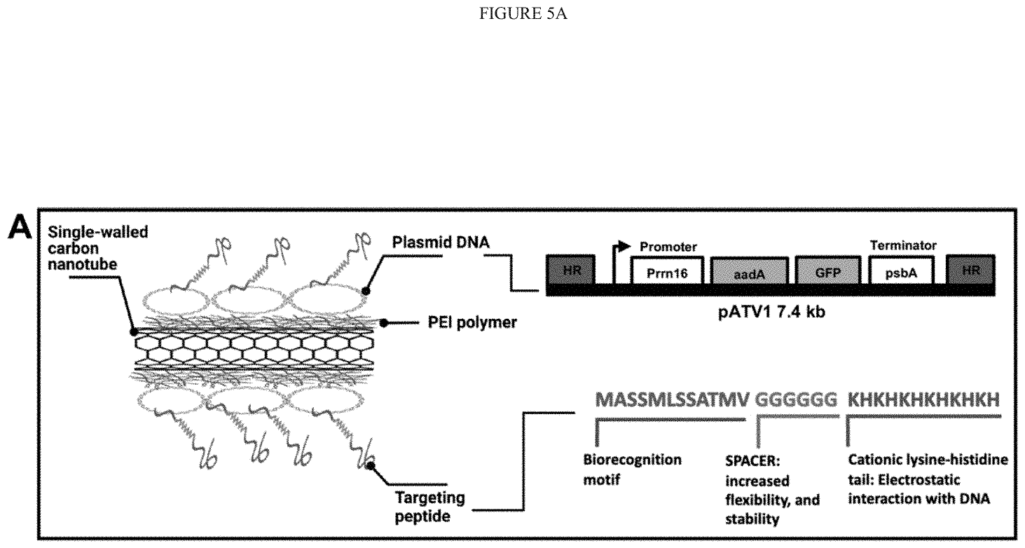

Polypeptides as described herein may be used to target nucleic acid(s) to the chloroplast of a plant. Accordingly, one or more polypeptides of the invention may be included in a conjugate comprising one or more nucleic acids. Such conjugates may further comprise a functionalized nanoparticle as described herein. The components present in a conjugate are generally bound via electrostatic interactions: a polypeptide of the invention is able to electrostatically bind to a nucleic acid; and the nucleic acid is able to electrostatically bind to a functionalized nanoparticle as described herein. Thus, a conjugate as described herein may comprise a functionalized nanoparticle electrostatically bound to one or more nucleic acid molecules and one or more polypeptides of the invention may be electrostatically bound to at least one of the nucleic acid molecules (i.e., each nucleic acid molecule bound to the functionalized nanoparticle may be electrostatically bound to 0, 1, 2, 3, or more polypeptides of the invention, provided that at least one nucleic acid molecule present in the conjugate is electrostatically bound to at least 1 such polypeptide) (see, e.g., FIG. 5A).

Accordingly, certain embodiments provide a conjugate comprising one or more polypeptides of the invention and a nucleic acid (e.g., a polynucleotide), wherein the one or more polypeptides are electrostatically bound to the nucleic acid (e.g., bound to the backbone of the nucleic acid).

Certain embodiments also provide a conjugate comprising one or more polypeptides of the invention, one or more nucleic acids (e.g., one or more polynucleotides), and a functionalized nanoparticle, wherein the one or more nucleic acids are electrostatically bound to the functionalized nanoparticle, and wherein the one or more polypeptides are electrostatically bound to the one or more nucleic acids (e.g., bound to the backbone of the nucleic acid).

As used herein, the term “conjugate” includes two or more elements that are combined, linked, associated, bound or joined either reversibly or irreversibly. For example, a conjugate may comprise two or more elements that are associated or bound to each other through an electrostatic interaction.

As used herein, the terms “electrostatic binding”, “electrostatically bound” and other similar variations, refer for a non-covalent bond between two or more molecules, wherein the molecules are associated/bound to each other through the force of attraction between charged molecules and/or particles. For example, such molecules may be bound via, e.g., ion pairing, dipole interactions, charge-dipole interactions, pi-interactions, and/or hydrogen bonding. In certain embodiments, a charged molecule (e.g., a polypeptide of the invention or a functionalized nanoparticle) may be attracted to the negatively charged backbone of a nucleic acid, thereby electrostatically binding the charged molecule and the nucleic acid. The electrostatic binding between two molecules may be reversible. For example, in certain embodiments, the electrostatic forces binding the elements comprised within a conjugate described herein are sufficient to keep the conjugate intact throughout delivery to the chloroplast; however, the elements in the conjugate may separate to the extent necessary for transcription of the nucleic acid upon delivery to the chloroplast.

As used herein, the term “nanoparticle” refers to a quantum dot, carbon dot, carbon nanotube (e.g., single walled carbon nanotube (SWCNT)), silica nanoparticle (e.g., porous silica nanoparticle), metal or metal oxide nanoparticle (e.g., gold, silver, copper, zinc, zinc oxide, magnesium, magnesium oxide, cerium oxide, or iron oxide nanoparticle).

As used herein, a “functionalized nanoparticle” refers to a nanoparticle having a surface that is capable of being linked or attracted (e.g. through electrostatic interactions) to a nucleic acid. In certain embodiments, the nanoparticle is a cationic nanoparticle, or the surface of a neutral or anionic nanoparticle is modified into a cationic surface that is capable of being attracted to a nucleic acid. For example, the functionalized nanoparticle surface may comprise amines that can become protonated. The protonated amines may be capable of interacting electrostatically with a negatively charged backbone of a nucleic acid molecule, thereby linking or associating the nucleic acid with the functionalized nanoparticle. In certain embodiments, the protonated amines are covalently bound to the nanoparticle directly or through a linker. In certain embodiments, the protonated amines are comprised within/part of a material that is capable of coating the nanoparticle to produce the functionalized nanoparticle. In certain embodiments, the coating material is a cationic material that comprises amine groups. For example, such a coating may comprise polyethyleneimine (PEI) and/or ethylenediamine (EDA), which comprise amines that are capable of being protonated. Thus, in certain embodiments, the functionalized nanoparticle has a coating that comprises PEI and/or EDA having amines, wherein some or all of the amines are protonated. In certain embodiments, the functionalized nanoparticle is coated with PEI having amines, wherein some or all of the amines are protonated. In certain embodiments, the functionalized nanoparticle is coated with EDA having amines, wherein some or all of the amines are protonated. In certain embodiments, the functionalized nanoparticle is coated with PEI and EDA having amines, wherein some or all of the amines are protonated.

As used herein, the term “coated” refers to when at least about 25% of the outer surface area of the nanoparticle is covered with the coating material. In certain embodiments, a nanoparticle is coated when at least about 30%, 40%, 50%, 60%, 70%, 80%, 90%, 95%, or 100% of the outer surface area of the nanoparticle is covered with the coating material. In certain embodiments, a nanoparticle is coated when at least about 50%, 60%, 70%, 80%, 90%, 95%, or 100% of the outer surface area of the nanoparticle is covered with the coating material.

In certain embodiments, the functionalized nanoparticle has a dimension in the range of about lnm to 1000 nm. For example, the nanostructure may have a longest dimension (e.g., diameter) in the range of about 1-900 nm, 1-800 nm, 1-700 nm, 1-600 nm, 1-500 nm, 1-400 nm, 1-300 nm, 1-250 nm, 1-200 nm, 1-150 nm, 1-100 nm, 1-90 nm, 1-80 nm, 1-70 nm, 1-60 nm, 1-50 nm, 1-40 nm, 1-30 nm, 1-25 nm, 1-20 nm, 1-15 nm, 1-10 nm or 1-5 nm. The “nanoparticle” may be approximately spherical, or non-spherical. Methods for characterizing nanoparticles are known in the art and are described herein (e.g., electron microscopy, dynamic light scattering, or atomic force microscopy).

In certain embodiments, the functionalized nanoparticle is a functionalized quantum dot.

In certain embodiments, the functionalized nanoparticle is a functionalized carbon dot.

In certain embodiments, the functionalized nanoparticle is a functionalized carbon nanotube. In certain embodiments, the functionalized nanoparticle is a functionalized single walled carbon nanotube (SWCNT)).

In certain embodiments, the functionalized nanoparticle is a functionalized silica nanoparticle (e.g., porous silica nanoparticle).

In certain embodiments, the functionalized nanoparticle is a functionalized metal or metal oxide nanoparticle (e.g., gold, silver, copper, zinc, zinc oxide, magnesium, magnesium oxide, cerium oxide, or iron oxide nanoparticle).

In certain embodiments, the conjugate comprises two or more polypeptides of the invention, which may be the same or different. In certain embodiments, a conjugate as described herein comprises at least about 2, 5, 10, 15, 20, 30, 40, 50, 60, 70, 80, 90, 100, 125, 150, 175, 200, 300, 400, 500 or more polypeptides as described herein. In certain embodiments, a conjugate as described herein comprises from between about 2 to 500; or 2 to 400; or 2 to 300; or 2 to 200; or 2 to 100; or 2 to 50; or 2 to 40; or 2 to 30; or 2 to 25; or 2 to 20; or 2 to 15; or 2 to 10 polypeptides of the invention. In certain embodiments, the two or more polypeptides are the same. In certain embodiments, the conjugate comprises a mixture of different polypeptides of the invention.

In certain embodiments, the conjugate comprises two or more nucleic acid molecules, which may be the same or different. In certain embodiments, the two or more nucleic acid molecules are the same. In certain embodiments, one or more nucleic acid molecules may be electrostatically bound to the functionalized nanoparticle. For example, one or more copies of a nucleic acid molecule (e.g., plasmid DNA) may be bound to the functionalized nanoparticle (e.g., PEI coated carbon nanotube). The number of nucleic acid molecules bound to the nanoparticle may vary, for example, depending on the size of the nucleic acid molecule and the size and/or surface charge of the functionalized nanoparticle. In certain embodiments, from about 1 to 96, 2 to 84, 3 to 72, 4 to 60, 5 to 48, 6 to 36, 7 to 24, or 8 to 12 nucleic acid molecules (e.g., plasmids) may be bound to the functionalized nanoparticle. In certain embodiments, about 1, 2, 3, 4, 5, 6, 7, 8, 9, 10, 15, 20, 30, 40, 50, 60, 70, 80, 90, or 100 nucleic acid molecules (e.g., plasmids) may be bound to the functionalized nanoparticle.

In certain embodiments, to achieve high surface binding, an excess amount (mass ratio or molar ratio) of nucleic acid relative to the functionalized nanoparticle may be provided for complexing with the functionalized nanoparticle. In certain embodiments, to achieve high targeting polypeptide binding, an excess amount (mass ratio or molar ratio) of polypeptide relative to nucleic acid may be provided for complexing with the functionalized nanoparticle bound nucleic acid. In certain embodiments, the mass ratio of the functionalized nanoparticle to nucleic acid to polypeptide in a conjugate described herein is about 1:2:50. In certain embodiments, the mass ratio of the functionalized nanoparticle to nucleic acid to polypeptide is about 1:1.5:30. In certain embodiments, the mass ratio of the functionalized nanoparticle to nucleic acid to polypeptide is about 1:2:50. In certain embodiments, the mass ratio of the functionalized nanoparticle to nucleic acid to polypeptide is about 1:3:60. In certain embodiments, the mass ratio of the functionalized nanoparticle to nucleic acid to polypeptide is about 1:5:80.

In certain embodiments, a nucleic acid for inclusion in a conjugate described herein is at least about 10, 20, 30, 40, 50, 60, 70, 80, 90, 100, 500, 1,000, 2,000, 3,000, 4,000, 5,000, 6000, 7000, 8000, 9000, 10000 or more nucleotides in length. In certain embodiments, the nucleic acid is between about 10 to about 10,000 nucleotides in length. In certain embodiments, the nucleic acid is between about 15 to about 9,000 nucleotides in length. In certain embodiments, the nucleic acid is between about 15 to about 7,500 nucleotides in length. In certain embodiments, the nucleic acid is between about 6,000 to about 9,000 nucleotides in length. In certain embodiments, the nucleic acid is between about 7,000 to about 8,000 nucleotides in length. In certain embodiments, the nucleic acid is between about 10 to about 50 nucleotides in length. In certain embodiments, the nucleic acid is between about 10 to about 30 nucleotides in length. In certain embodiments, the nucleic acid is between about 10 to about 20 nucleotides in length.

In certain embodiments, the nucleic acid is DNA. In certain embodiments, the nucleic acid is cDNA. In certain embodiments, the nucleic acid is RNA (e.g., siRNA or miRNA). In certain embodiments, the nucleic acid is a DNA/RNA hybrid.

In certain embodiments, the nucleic acid is an expression cassette comprising a promoter operably linked to a nucleic acid sequence of interest (e.g., encoding a target protein of interest). In certain embodiments, the nucleic acid is an expression cassette comprising a chloroplast-specific transcriptional regulation sequence(s) operably linked to a nucleic acid sequence of interest. For example, in certain embodiments, the expression cassette comprises a chloroplast-specific promoter. In certain embodiments, the expression cassette comprises a plastid ribosomal RNA operon (prrn) promoter, which can be specifically transcribed by the chloroplast expression machinery (e.g., Nicotiana tabacum Prrn or Prrn16 element) (see, e.g., Suzuki et al., (2003), Plant Cell, 15:195-205). In certain embodiments, the expression cassette further comprises a selection marker (e.g., an antibiotic resistance gene). In certain embodiments, the expression cassette further comprises a spectinomycin (aaDa) selection marker. In some embodiments, the expression cassette also includes additional sequences for expression fidelity. For example, expression cassette may further comprise a sequence encoding a 5′ untranslated region for mRNA stability (e.g., comprises a T7g10 5′ untranslated region sequence; see, e.g., Svab et al., (1990) PNAS, 87(21):8526-8530; Maliga & Bock, (2011), Plant Physiology, 155:1501-1510). In certain embodiments, the expression cassette further comprises a terminator region sequence (e.g., Nt psbA).

In certain embodiments, the nucleic acid is a DNA plasmid. Such a plasmid may comprise an expression cassette described herein. In certain embodiments, the plasmid comprises homologous recombination sites flanking the expression cassette, which may enable insertion into the inverted repeat region of the plastid genome.

In certain embodiments, the nucleic acid encodes an RNA molecule or a target protein. In certain embodiments, the nucleic acid encodes a fluorescent protein (e.g., GFP).

In certain embodiments, one or more of the nucleic acids comprised in a conjugate are linked (covalently linked) to a fluorescent molecule, such as Cy3.

Certain embodiments provide a conjugate of the invention comprising one or more polypeptides of the invention, one or more nucleic acids and a functionalized nanoparticle, wherein the conjugate is produced by combining one or more nucleic acids with the functionalized nanoparticle under conditions suitable to produce a nucleic acid-functionalized nanoparticle conjugate; and combining the nucleic acid-functionalized nanoparticle conjugate with one or more polypeptides of the invention under conditions suitable to produce the conjugate of the invention. In certain embodiments, the process further comprises combining a coating material that comprises amines that are capable of being protonated (e.g., PEI and/or EDA) with a nanoparticle under conditions suitable for the coating material to covalently bind to the outer surface of the nanoparticle to produce the functionalized nanoparticle.

### Compositions

Certain embodiments provide a composition comprising a polypeptide as described herein and a carrier.

Certain embodiments also provide a conjugate as described herein and a carrier.

Certain embodiments also provide a composition comprising one or more polypeptides as described herein, one or more nucleic acids and a functionalized nanoparticle.

Such compositions may be further formulated into a suitable dosage form for plant application/introduction.

Common agrochemical and/or nanomaterial formulations are known in the field and include liquid and solid formulations. Exemplary formulations include gel, aqueous or oil-based solutions, dispersions, suspensions or emulsions, such as those described in U.S. Pat. Nos. 5,139,152; 6,403,529; 6,878,674; 7,094,831; 7,109,267 and 9,706,771.

In certain embodiments, the composition is a liquid formulation, which may be administered or sprayed onto a plant using, e.g., ground/aerial spraying.

In certain embodiments, the composition is in a pellet or tablet form. Such formulations may be capable of rapid break-up in water using minimal or no agitation while providing fine dispersions of the active ingredient (see, e.g., U.S. Pat. Nos. 5,180,587 and 7,550,156).

In certain embodiments, the composition comprises agriculturally acceptable additives or excipients. Suitable additives or excipients which may be present in the formulations include organic solvents, solubilizers, emulsifiers, surfactants, dispersants, preservatives, colorants, fillers, diluents, binders, glidants, lubricants, disintegrants, antiadherents, sorbents, coatings, wetting agents, penetrants and vehicles. Well known additives, excipients and agrochemical formulations are described in U.S. Pat. No. 6,602,823 and the aforementioned US Patents.

In certain embodiments, the composition is in a powder dosage form.

In certain embodiments, the composition is in a lyophilized form and can be readily reconstituted into liquid form before use.

In certain embodiments, a conjugate as described herein is delivered or is capable of being delivered to a plant (e.g., via foliar application) at a concentration of about 0.001 mg/L, 0.01 mg/L, 0.01 mg/L, 0.1 mg/L, 1 mg/L, 2 mg/L, 3 mg/L, 4 mg/L, 5 mg/L, 6 mg/L, 7 mg/L, 8 mg/L, 9 mg/L, 10 mg/L, 15 mg/L, 20 mg/L, 30 mg/L, 40 mg/L, 50 mg/L, 60 mg/L, 70 mg/L, 80 mg/L, 90 mg/L, 100 mg/L, 200 mg/L, 300 mg/L, 400 mg/L, 500mg/L, 600 mg/L, 700 mg/L, 800 mg/L, 900 mg/L, 1000mg/L, 2000 mg/L, 3000 mg/L, 4000 mg/L, or 5000mg/L. In certain embodiments, a conjugate as described herein is delivered or is capable of being delivered to a plant (e.g., via foliar application) at a concentration of about less than 2 mg/L, 3 mg/L, 4 mg/L, 5 mg/L, 6 mg/L, 7 mg/L, 8 mg/L, 9 mg/L, 10 mg/L, 15 mg/L, 20 mg/L, 30 mg/L, 40 mg/L, or 50 mg/L.

In certain embodiments, a conjugate comprising single walled carbon nanotube (SWCNT) as described herein is delivered or is capable of being delivered to a plant (e.g., via foliar application) at a concentration of about 0.001 mg/L, 0.01 mg/L, 0.1 mg/L, 1 mg/L, 2 mg/L, 3 mg/L, 4 mg/L, 5 mg/L, 6 mg/L, 7 mg/L, 8 mg/L, 9 mg/L, 10 mg/L, 15 mg/L, or 20 mg/L. In certain embodiments, a conjugate comprising single walled carbon nanotube (SWCNT) as described herein is delivered or is capable of being delivered to a plant (e.g., via foliar application) at a concentration of no more than 5 mg/L or 2 mg/L, for example, without reducing plant cell viability (e.g., less than 10% plant cell death by day 5 after delivery or no difference as compared to a control by day 5 after delivery). In certain embodiments, a conjugate comprising single walled carbon nanotube (SWCNT) as described herein is delivered or is capable of being delivered to a plant (e.g., via foliar application) at a concentration of about 0.001 mg/L to 5 mg/L or about 0.01 mg/L to 3 mg/L (e.g., 2 mg/L).

In certain embodiments, a conjugate comprising carbon dot (CD) as described herein is delivered or capable of being delivered to a plant (e.g., via foliar application) at a concentration of about 0.001 mg/L, 0.01 mg/L, 0.1 mg/L, 1 mg/L, 2 mg/L, 3 mg/L, 4 mg/L, 5 mg/L, 6 mg/L, 7 mg/L, 8 mg/L, 9 mg/L, 10 mg/L, 15 mg/L, 20 mg/L, 30 mg/L, 40 mg/L, 50 mg/L, 60 mg/L, 70 mg/L, 80 mg/L, 90 mg/L, 100 mg/L, or 200 mg/L. In certain embodiments, a conjugate comprising carbon dot (CD) as described herein is delivered or is capable of being delivered to a plant (e.g., via foliar application) at a concentration of no more than 30 mg/L or 20 mg/L, for example, without reducing plant cell viability (e.g., less than 10% plant cell death by day 5 after delivery or no difference as compared to a control by day 5 after delivery). In certain embodiments, a conjugate comprising carbon dot (CD) as described herein is delivered or is capable of being delivered to a plant (e.g., via foliar application) at a concentration of about 0.001 mg/L to 40 mg/L or about 0.01 mg/L to 30 mg/L (e.g., 20 mg/L).

In certain embodiments, a conjugate (e.g., comprising single walled carbon nanotube (SWCNT) or carbon dot (CD) as described herein) is delivered or is capable of being delivered to a plant (e.g., via foliar application) at a concentration of about 0.001 mg/L to 2 mg/L or 0.001 mg/L to 20 mg/L (e.g., 2 or 20 mg/L), without increasing leaf H₂O₂ levels (e.g., by day 5 after delivery) as compared to a control.

In certain embodiments, a conjugate (e.g., comprising single walled carbon nanotube (SWCNT) or carbon dot (CD) as described herein) is delivered or is capable of being delivered to a plant (e.g., via foliar application) at a concentration of about 0.001 mg/L to 2 mg/L or 0.001 mg/L to 20 mg/L (e.g., 2 or 20 mg/L), without increasing chloroplast DNA oxidative damage level (e.g., no difference in the chloroplast 8-OHdG level by day 5 after delivery) as compared to a control.

In certain embodiments, a conjugate (e.g., comprising single walled carbon nanotube (SWCNT) or carbon dot (CD) as described herein) is delivered or is capable of being delivered to a plant (e.g., via foliar application) at a concentration of about 0.001 mg/L to 2 mg/L or 0.001 mg/L to 20 mg/L (e.g., 2 or 20 mg/L), without decreasing chloroplast intactness (e.g., no difference as observed by differential interference contrast (DIC) microscopy by day 5 after delivery) as compared to a control.

In certain embodiments, a conjugate (e.g., comprising single walled carbon nanotube (SWCNT) or carbon dot (CD) as described herein) is delivered or is capable of being delivered to a plant (e.g., via foliar application) at a concentration of about 0.001 mg/L to 2 mg/L or 0.001 mg/L to 20 mg/L (e.g., 2 or 20 mg/L), without decreasing maximum quantum yield of photosystem II (PSII) (e.g., no difference in Fᵥ/Fₘ by day 5 after delivery) as compared to a control.

### Certain Methods

Certain embodiments provide a method of delivering one or more nucleic acid molecules (e.g., one or more polynucleotides) to a chloroplast of a plant, the method comprising introducing to the plant (e.g., a leaf) a conjugate as described herein.

Certain embodiments provide a method of expressing a target RNA or protein in the chloroplast of a plant, the method comprising introducing to the plant (e.g., a leaf) a conjugate as described herein, wherein the conjugate comprises one or more nucleic acid molecules encoding the target RNA or protein.

Certain embodiments also provide a method of treating a disease in a plant, the method comprising introducing to the plant (e.g., a leaf) an effective amount of a conjugate as described herein, wherein the conjugate comprises a nucleic acid molecule effective to treat the disease.

In certain embodiments, introducing the conjugate to the plant comprises contacting a leaf of the plant. In certain embodiments, introducing the conjugate to the plant comprises contacting the top surface of a leaf of the plant.

In certain embodiments, the delivered conjugate is more efficiently introduced into a chloroplast of the plant as compared to a conjugate comprising a nucleic acid that is not linked to a polypeptide as described herein.

In certain embodiments, the plant is a dicotyledon plant. In certain embodiments, the plant is selected from the group consisting soybean, cotton, arabidopsis, citrus, tomato, and grapevine.

### Certain Embodiments

Embodiment 1. A polypeptide comprising a chloroplast-targeting amino acid sequence linked to an amino acid sequence that is capable of electrostatically binding to a nucleic acid molecule.

Embodiment 2. The polypeptide of embodiment 1, wherein the chloroplast-targeting amino acid sequence comprises an amino acid sequence of a ribulose biosphosphate carboxylase small chain 1A (RBCS1A) protein.

Embodiment 3. The polypeptide of embodiment 2, wherein the chloroplast-targeting amino acid sequence comprises an amino acid sequence of a dicot RBCS1A protein.

Embodiment 4. The polypeptide of embodiment 1, wherein the chloroplast-targeting amino acid sequence comprises an amino acid sequence having at least about 75%, 80% or 90% sequence identity to MASSMLSSATMV (SEQ ID NO:1).

Embodiment 5. The polypeptide of embodiment 4, wherein the chloroplast-targeting amino acid sequence comprises MASSMLSSATMV (SEQ ID NO:1).

Embodiment 6. The polypeptide of embodiment 4, wherein the chloroplast-targeting amino acid sequence consists of MASSMLSSATMV (SEQ ID NO:1).

Embodiment 7. The polypeptide of any one of embodiments 1-6, wherein the amino acid sequence that is capable of electrostatically binding to a nucleic acid molecule is lysine- and histidine-rich.

Embodiment 8. The polypeptide of embodiment 7, wherein the amino acid sequence that is capable of electrostatically binding to a nucleic acid molecule comprises an amino acid sequence having at least about 75%, 80%, 85%, 90%, or 95% sequence identity to

(SEQ ID NO: 2)
KHKHKHKHKHKH.

Embodiment 9. The polypeptide of embodiment 7, wherein the amino acid sequence that is capable of electrostatically binding to a nucleic acid molecule comprises

(SEQ ID NO: 2)
KHKHKHKHKHKH.

Embodiment 10. The polypeptide of embodiment 7, wherein the amino acid sequence that is capable of electrostatically binding to a nucleic acid molecule consists of

(SEQ ID NO: 2)
KHKHKHKHKHKH.

Embodiment 11. The polypeptide of any one of embodiments 1-10, wherein the chloroplast-targeting amino acid sequence is linked to the amino acid sequence that is capable of electrostatically binding to a nucleic acid molecule by a peptide bond or by a polypeptide linker.

Embodiment 12. The polypeptide of embodiment 11, wherein the chloroplast-targeting amino acid sequence is linked to the amino acid sequence that is capable of electrostatically binding to a nucleic acid by a peptide bond.

Embodiment 13. The polypeptide of embodiment 11, wherein the chloroplast-targeting amino acid sequence is linked to the amino acid sequence that is capable of electrostatically binding to a nucleic acid by a polypeptide linker.

Embodiment 14. The polypeptide of embodiment 13, wherein the polypeptide linker is a flexible polypeptide linker.

Embodiment 15. The polypeptide of embodiment 13 or 14, wherein the polypeptide linker is a glycine-rich linker.

Embodiment 16. The polypeptide of embodiment 15, wherein the polypeptide linker is GGGGGG (SEQ ID NO:3).

Embodiment 17. The polypeptide of embodiment 1, which comprises an amino acid sequence having at least about 75%, 80%, 85%, 90%, or 95% sequence identity to

(SEQ ID NO: 4)
MASSMLSSATMVGGGGGGKHKHKHKHKHKH.

Embodiment 18. The polypeptide of embodiment 17, which comprises

(SEQ ID NO: 4)
MASSMLSSATMVGGGGGGKHKHKHKHKHKH.

Embodiment 19. The polypeptide of embodiment 17, which consists of

(SEQ ID NO: 4)
MASSMLSSATMVGGGGGGKHKHKHKHKHKH.

Embodiment 20. A polynucleotide comprising a nucleic acid sequence encoding the polypeptide of any one of embodiments 1-19.

Embodiment 21. A conjugate comprising one or more polypeptides as described in any one of embodiments 1-19 and a nucleic acid, wherein the one or more polypeptides are electrostatically bound to the nucleic acid.

Embodiment 22. A conjugate comprising one or more polypeptides as described in any one of embodiments 1-19, one or more nucleic acids, and a functionalized nanoparticle, wherein the one or more nucleic acids are electrostatically bound to the functionalized nanoparticle, wherein the one or more polypeptides are electrostatically bound to the one or more nucleic acids, and wherein the functionalized nanoparticle is selected from the group consisting of a functionalized quantum dot, carbon dot, carbon nanotube, silica nanoparticle, and metal or metal oxide nanoparticle.

Embodiment 23. The conjugate of embodiment 21 or 22, wherein the nucleic acid is DNA.

Embodiment 24. The conjugate of any one of embodiments 21-23, wherein the nucleic acid is a DNA plasmid.

Embodiment 25. The conjugate of any one of embodiments 21-24, wherein the nucleic acid encodes an RNA molecule or a target protein.

Embodiment 26. The conjugate of any one of embodiments 22-25, wherein the functionalized nanoparticle is coated with polyethyleneimine (PEI) and/or ethylenediamine (EDA), which comprise amines that are capable of being protonated.

Embodiment 27. The conjugate of embodiment 26, wherein the functionalized nanoparticle is coated with PEI.

Embodiment 28. The conjugate of any one of embodiments 22-27, wherein the functionalized nanoparticle is a single walled carbon nanotube (SWNT).

Embodiment 29. A composition comprising the polypeptide of any one of embodiments 1-19 or the conjugate of any one of embodiments 21-28 and a carrier.

Embodiment 30. A method of delivering one or more nucleic acids to a chloroplast of a plant, the method comprising introducing to the plant a conjugate as described in any one of embodiments 21-28.

Embodiment 31. A method of expressing a target RNA or protein in the chloroplast of a plant, the method comprising introducing to the plant (e.g., a leaf) a conjugate as described in any one of embodiments 21-28, wherein the conjugate comprises one or more nucleic acids encoding the target RNA or protein.

Embodiment 32. A method of treating a disease in a plant, the method comprising introducing to the plant (e.g., a leaf) an effective amount of a conjugate as described in any one of embodiments 21-28, wherein the conjugate comprises one or more nucleic acids effective to treat the disease.

Embodiment 33. The method of any one of embodiments 30-32, wherein the plant is a dicotyledon plant selected from the group consisting of soybean, cotton, arabidopsis, citrus, tomato, and grapevine.

### Certain Definitions

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucl. Acids Res.,19:5081; Ohtsuka et al. (1985) JBC, 260(5):2605-2608; Rossolini et al. (1994) Mol. Cell. Probes, 8(2):91-98). A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

By “portion” or “fragment,” as it relates to a nucleic acid molecule, sequence or segment of the invention, when it is linked to other sequences for expression, is meant a sequence having at least 80 nucleotides, more preferably at least 150 nucleotides, and still more preferably at least 400 nucleotides. If not employed for expressing, a “portion” or “fragment” means at least 9, preferably 12, more preferably 15, even more preferably at least 20, consecutive nucleotides, e.g., probes and primers (oligonucleotides), corresponding to the nucleotide sequence of the nucleic acid molecules of the invention.

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆) alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein).

The terms “protein,” “peptide” and “polypeptide” are used interchangeably herein. Polypeptide sequences specifically recited herein are written with the amino terminus on the left and the carboxy terminus on the right.

The invention encompasses isolated or substantially purified nucleic acid or protein compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

“Wild-type” refers to the normal gene, or organism found in nature without any known mutation.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

“Recombinant DNA molecule” is a combination of DNA sequences that are joined together using recombinant DNA technology and procedures used to join together DNA sequences as described, for example, in Sambrook and Russell, (2001). Molecular cloning: A laboratory manual 3rd Ed. 620 Cold Spring Harbor Laboratory Press. Plainview, N.Y., 621.

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

The term “gene” is used broadly to refer to any segment of nucleic acid associated with a biological function. Genes include coding sequences and/or the regulatory sequences required for their expression. For example, gene refers to a nucleic acid fragment that expresses mRNA, functional RNA, or a specific protein, including its regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

A “vector” is defined to include, inter alia, any viral vector, plasmid, cosmid, phage or binary vector in double or single stranded linear or circular form which may or may not be self-transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and plant promoters (e.g., chloroplast-specific promoters).

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al. (1995) Mol. Biotech. 3(3):225-236).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA- box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

As used herein, the term “operably linked” refers to a linkage of two elements in a functional relationship. For example, “operably linked” may refer to a linkage of polynucleotide elements or polypeptide elements in a functional relationship. A nucleic acid is “operably linked” when it is placed into a functional relationship with another nucleic acid sequence. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. “Operably-linked” also refers to the association two chemical moieties so that the function of one is affected by the other, e.g., an arrangement of elements wherein the components so described are configured so as to perform their usual function.

“Expression” refers to the transcription and/or translation in a cell of an endogenous gene, transgene, as well as the transcription and stable accumulation of sense (mRNA) or functional RNA. In the case of antisense constructs, expression may refer to the transcription of the antisense DNA only. Expression may also refer to the production of protein.

“Transcription stop fragment” refers to nucleotide sequences that contain one or more regulatory signals, such as polyadenylation signal sequences, capable of terminating transcription. Examples of transcription stop fragments are known to the art.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

The following terms are used to describe the sequence relationships between two or more sequences (e.g., nucleic acids, polynucleotides or polypeptides): (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA, gene sequence or peptide sequence, or the complete cDNA, gene sequence or peptide sequence.

(b) As used herein, “comparison window” makes reference to a contiguous and specified segment of a sequence, wherein the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller (1988) CABIOS, 4(1):11-17; the local homology algorithm of Smith et al. (1981) Adv. Appl. Math. 2(4):482-489; the homology alignment algorithm of Needleman and Wunsch, (1970) JMB, 48(3):443-453; the search-for-similarity-method of Pearson and Lipman, (1988) Proc. Natl. Acad. Sci. USA, 85(8):2444-2448; the algorithm of Karlin and Altschul, (1990) Proc. Natl. Acad. Sci. USA, 87(6):2264-2268, modified as in Karlin and Altschul, (1993) Proc. Natl. Acad. Sci. USA, 90(12):5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73(1):237-244; Higgins et al. (1989) CABIOS 5(2):151-153; Corpet et al. (1988) Nucl. Acids Res. 16(22):10881-10890; Huang et al. (1992) CABIOS 8(2):155-165; and Pearson et al. (1994) Meth. Mol. Biol. 24:307. The ALIGN program is based on the algorithm of Myers and Miller, supra. The BLAST programs of Altschul et al. (1990) JMB, 215(3):403-410; Altschul et al. Nucl. Acids Res., 25(17):3389-3402 (1997), are based on the algorithm of Karlin and Altschul supra.

Software for performing BLAST analyses is publicly available through the National Center for Biotechnology Information (available on the world wide web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al. (1997) Nucleic Acids Res. 25(17):3389-3402. Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world wide web at ncbi.nlm.nih.gov. Alignment may also be performed manually by visual inspection.

For purposes of the present invention, comparison of sequences for determination of percent sequence identity to another sequence may be made using the Blast program (e.g., BlastN, version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. Optimal alignment is conducted using the homology alignment algorithm of Needleman and Wunsch, J. Mol. Biol. 48(3):443-453 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may results form, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel (1985) Proc. Natl. Acad. Sci. USA 82(2):488-492; Kunkel et al. (1987) Meth. Enzymol. 154:367; U.S. Pat. No. 4,873,192; Walker and Gaastra (1983) Techniques in Mol. Biol. (MacMillan Publishing Co., and the references cited therein; Walker, et al., (2012). Techniques in Molecular Biology. Springer Science & Business Media. Guidance as to appropriate amino acid substitutions that do not affect biological activity of the protein of interest may be found in the model of Dayhoff et al., Atlas of Protein Sequence and Structure, Supplement (Natl. Biomed. Res. Found. 1978) (see also, Dayhoff, (1969). Atlas of Protein Sequence and Structure. National Biomedical Research Foundation). Conservative substitutions, such as exchanging one amino acid with another having similar properties, are preferred. Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass naturally occurring proteins as well as variations and modified forms thereof. Such variants will continue to possess the desired activity. In certain embodiments, the deletions, insertions, and substitutions of the polypeptide sequence encompassed herein may not produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions or additions which alter, add or delete a single amino acid or a small percentage of amino acids in an encoded sequence are also “conservatively modified variations.”

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” “transduced” and “recombinant” refer to a host cell or organism into which a heterologous nucleic acid molecule has been introduced. The nucleic acid molecule can be stably integrated into the genome generally known in the art and are disclosed in Sambrook and Russell, supra. See also, Innis et al., PCR Protocols, Academic Press (1995); Wetmur, et al., (1995). PCR strategies. Academic Press New York, NY; Innis and Gelfand, PCR Methods Manual, Academic Press (1999); Innis, et al., (1995). PCR Strategies. Elsevier. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

“Genetically altered cells” denotes cells which have been modified by the introduction of recombinant or heterologous nucleic acids (e.g., one or more DNA constructs or their RNA counterparts) and further includes the progeny of such cells which retain part or all of such genetic modification.

As used herein, the term “derived” or “directed to” with respect to a nucleotide molecule means that the molecule has complementary sequence identity to a particular molecule of interest.

The terms “introduce” and “introduction” refers to contacting a plant, or a portion thereof, with a material (e.g., a composition described herein). For example, a composition as described herein may be applied to the plant, or a portion thereof (e.g., leaf). In certain embodiments, the plant or a portion thereof (e.g., foliage and/or other tissues) is sprayed with the composition. In certain embodiments, the plant or a portion thereof is coated with the composition (e.g., a leaf dipped in a composition). In certain embodiments, the composition is administered to the plant (e.g., via injection).

The terms “treat” and “treatment” refer to both therapeutic treatment and prophylactic or preventative measures, wherein the object is to prevent or decrease an undesired change, condition or disease in a plant. For purposes of this invention, beneficial or desired results include, but are not limited to, alleviation of symptoms, diminishment of extent of disease, stabilized (i.e., not worsening) state of disease, delay or slowing of disease progression or transmission, and amelioration of the disease state, whether detectable or undetectable. A plant in need thereof treatment include plants already with the condition or disease as well as those prone to have the condition or disease or those in which the condition or disease is to be prevented.

The phrase “effective amount” means an amount of a composition as described herein that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The invention will now be illustrated by the following non-limiting Example.

### EXAMPLE 1 TARGETED CARBON NANOSTRUCTURES FOR NUCLEIC ACID DELIVERY TO PLANT CHLOROPLASTS

Nanotechnology approaches for improving the delivery efficiency of chemicals and molecular cargoes in plants through plant biorecognition mechanisms remain relatively unexplored. In this Example, we developed targeted carbon-based nanomaterials as tools for precise chemical delivery (carbon dots, CDs) and gene delivery platforms (single-walled carbon nanotubes, SWCNTs) to chloroplasts, key organelles involved in efforts to improve plant photosynthesis, assimilation of nutrients, and delivery of agrochemicals. A biorecognition approach of coating the nanomaterials with a rationally designed chloroplast targeting peptide improved the delivery of CDs with molecular baskets (TP-β-CD) for delivery of agrochemicals and of plasmid DNA coated SWCNT (TP-pATV1-SWCNT) from 47% to 70% and from 39% to 57% of chloroplasts in leaves, respectively. Plants treated with TP-β-CD (20 mg/L) and TP-pATV1-SWCNT (2 mg/L) had a low percentage of dead cells, 6% and 8%, respectively, similar to controls without nanoparticles, and no permanent cell and chloroplast membrane damage after 5 days of exposure. However, targeted nanomaterials transiently increased leaf H₂O₂ (0.3225 μmol gFW-1) above control plant levels (0.03441 μmol gFW-1) but within the normal range reported in land plants. The increase in leaf H₂O₂ levels was associated with oxidative damage in whole plant cell DNA, a transient effect on chloroplast DNA, and a decrease in leaf chlorophyll content (−17%) and carbon assimilation rates at saturation light levels (−32%) with no impact on photosystem II quantum yield. This work provides targeted delivery approaches for carbon-based nanomaterials mediated by biorecognition and a comprehensive understanding of their impact on plant cell and molecular biology for engineering safer and efficient agrochemical and biomolecule delivery tools.

### Introduction

The increasing demand for food production requires innovative and sustainable technologies for efficient agrochemical and biomolecule delivery in plants. Human population growth is expected to require a 60% increase or more in food production by 2050 relative to 2005-2007 levels.¹ Traditional plant breeding, genetic engineering, and land management strategies are not on track to meet the need for increased food production.^{2,3} Climate change will further complicate efforts toward achieving food security by exacerbating the frequency and intensity of environmental stresses that negatively impact crop productivity.^{4,5} The higher demand for higher crop yields is straining the earth's ecosystems by increasing energy, water, land use, and environmental pollution.^{6,7} The transition to sustainable food production systems will require innovations in agrochemical delivery and genetic engineering strategies. Nanotechnology is emerging as a tool to improve sustainable agricultural practices and maintain food security during a rapidly increasing human population and the threat of climate change impact on crop yields.⁸⁻¹⁰

Nanotechnology is providing approaches for more precise agrochemical delivery, genetic engineering platforms, and environmental sensing for enabling farmers to monitor, manage, and improve crop productivity.^{3,8,10-13} The use of engineered nanomaterials in agriculture may benefit from both advancing our fundamental understanding of nanomaterial-plant interactions and elucidating the impact of nanomaterials on plant function. Nanotechnology applications without adequately evaluating the biological impact on plant function can lead to unforeseen plant health and environmental consequences, causing decreased crop yield and pollution of the environment.^{10,14,15} Therefore, studies on the design of nanomaterials should go hand in hand with research on their biocompatibility with plants.

Nanomaterials exhibit tunable physical and chemical properties such as size, surface charge, amphiphilicity, and biomolecule coatings that enable targeted and controlled delivery of chemicals and biomolecules.¹⁶⁻²⁰ Targeted delivery through nanomaterials in agriculture has gained interest due to its tremendous potential for improving pesticide, herbicide, and fertilizer delivery while decreasing the environmental impact due to agrochemical runoff.^{3,9,10,16,21} Current approaches for improving the delivery efficiency of nanomaterials and their cargoes in plants have been based on modifications of the nanoparticle charge, size and hydrophobicity.^{18-20,22-24} The use of biomolecule coatings to guide nanomaterials to plant cells and organelles by the plant biorecognition machinery has only been recently explored and remains poorly understood.^{16,26} For example, Santana et al. recently demonstrated that quantum dots functionalized with a highly conserved chloroplast targeting peptide among dicot plants could deliver nanomaterials with chemical cargoes inside ˜75% of chloroplasts in leaf cells and modulate chloroplast redox status.^{16,26} These CdSe quantum dots were used for fundamental research on plant-nanoparticle interactions because long-term exposure to cadmium-based nanomaterials can be toxic to plants and the environment.²⁷ Quantum dots are model nanomaterials for understanding plant-nanoparticle interactions that can be traced by multiple advanced analytical tools including confocal fluorescence microscopy and elemental analysis, but they are not suitable for nanoenabled agriculture applications due to their toxicity.^{27,28} In contrast, carbon dots (CDs) are among the most biocompatible²⁹⁻³¹ and degradable nanomaterials^{31,32} made from renewable resources such as citric acid and urea.^{18,33} Functionalizing CDs with molecular baskets (β-cyclodextrins) able to carry a wide range of agrochemicals^{16,34,35} and targeting peptides allow them to act as sustainable targeted chemical delivery tools for agricultural applications.

Nanomaterials are also promising genetic engineering platforms due to their ability to bypass plant cell barriers including the cell wall and lipid membranes without mechanical aid in a broad array of plant species, including some recalcitrant to conventional genetic engineering approaches.^{9,11,19,24,36} High aspect ratio nanomaterials functionalized with polymers have been reported to enable the delivery of genetic elements into plants nuclear and chloroplast genomes.^{11,19,37,38} The delivery of a DNA plasmid encoding a green fluorescent protein (GFP) to the plant nuclear genome was mediated by single-walled carbon nanotubes (SWCNTs) that were covalently modified with a cationic polymer (polyethylenimine, PEI).^{24,37} The surface functionalization with PEI allowed interactions with plasmid DNA cargoes and the transport across plant cell barriers including the cell wall and plasma membrane. Passive delivery of plasmid DNA without mechanical aid was confirmed by expression analysis of GFP using digital drop PCR and confocal imaging in live plant cells. Furthermore, Kwak et al. reported the delivery of plasmid DNA encoding a yellow fluorescent protein into chloroplasts by chitosan-coated SWCNTs and assessed expression by confocal microscopy analysis.¹⁹ These studies investigated plasmid DNA delivery to chloroplasts by tuning the SWCNT nanomaterial surface condition with polymers. However, biorecognition approaches for targeting plasmid DNA via SWCNTs are needed. There is also a need to determine the biocompatibility of these carbon nanostructures and their impact on plant cell and organelle function for plant genetic engineering applications.

Studies on CDs and SWCNTs impact on plants and the environment tend to be performed after plant exposure to high doses of nanomaterials, >100 mg/L and >25 mg/L, respectively.^{14,39} The studies on interactions between carbon-based nanomaterials and plants have mainly focused on nanomaterials delivered through hydroponic, soil, and agar substrates^{29,40,41} but not by foliar delivery approaches. Mechanistic studies might be needed to determine the effect of targeted carbon nanomaterials within a range of concentrations intended for plant biotechnology and agricultural systems.^{39,42} Biological impact studies may provide insights into designs and synthesis of nanomaterials that do not negatively affect plant growth and development.³⁹ Understanding the effect that targeted nanomaterials have on plant cell and molecular biology could be useful toward engineering safer and effective chemical and biomolecule delivery strategies.

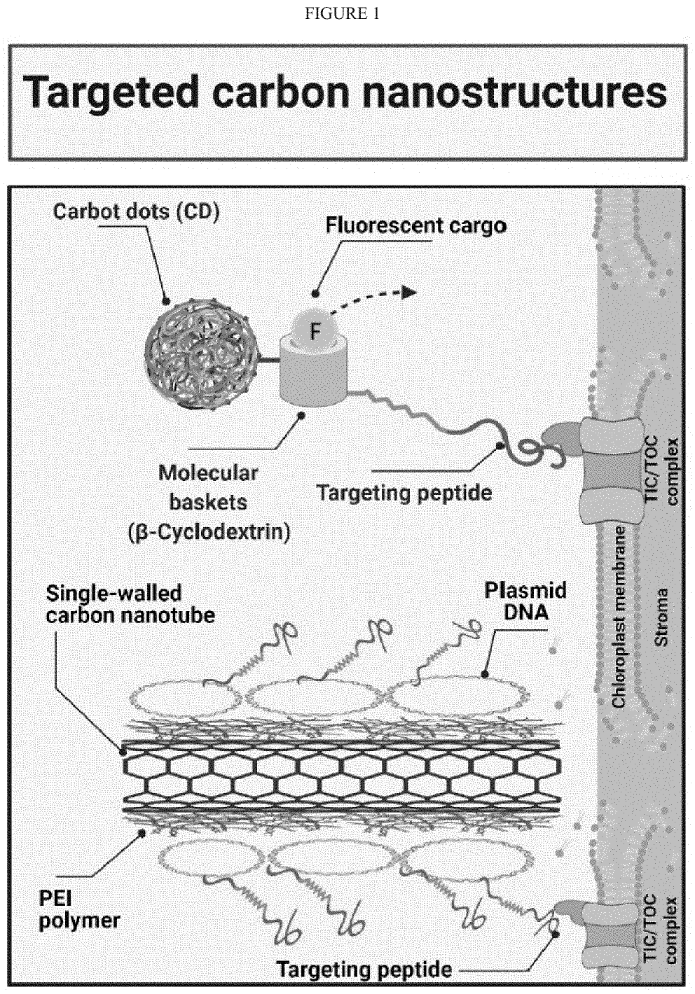

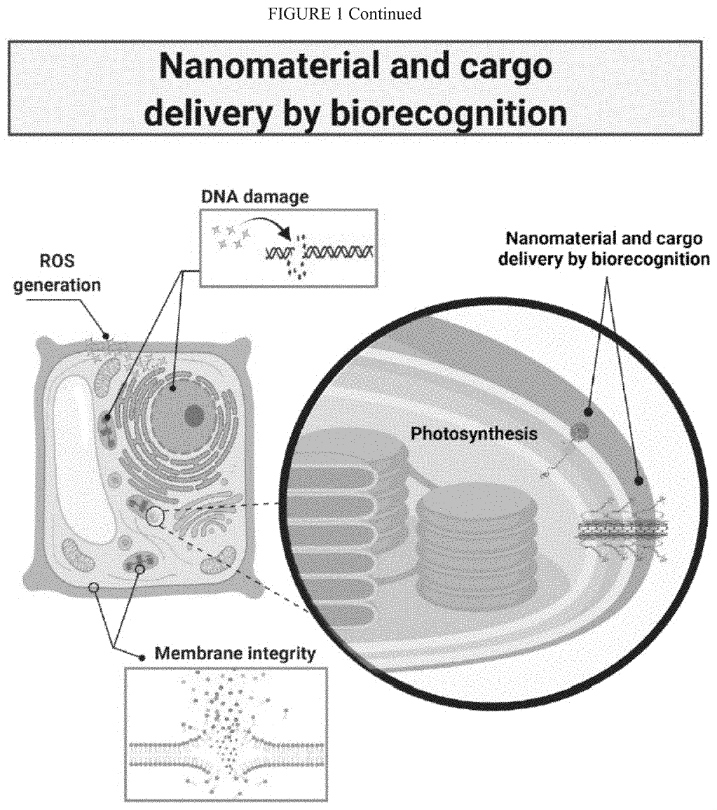

This Example developed nanocarriers for targeted delivery of chemicals and plasmid DNA to chloroplasts using carbon-based nanomaterials and investigated the impact of these nanocarriers on plant cell and molecular biology (FIG. 1). The targeted CDs contained a β-cyclodextrin molecular basket able to form inclusion complexes with chemical cargos^{16,34,35} and a targeting peptide (TP) from the rubisco small subunit 1A that improves binding and uptake by chloroplasts (TP-β-CD). The SWCNT functionalized with cationic polymers bound to plasmid DNA driven by a plastid-specific promoter (pATV1)⁴³ and to a chloroplast targeting peptide are shown to act as a targeted gene delivery platform (TP-pATV1-SWCNT). We show proof of concept that these carbon nanostructures target the delivery of a fluorescent chemical cargo and plasmid DNA through confocal fluorescence microscopy and molecular analysis. We investigated the impact of targeted nanomaterials on percentage of viable leaf cells, cell and plastid membrane intactness, leaf cell H₂O₂ levels, oxidative damage to DNA, chlorophyll, and photosynthesis. This work provides a comprehensive understanding of the interactions of targeted carbon nanostructures with cargoes in plants and their impact on plant cell, organelle, and molecular biology.

### Results and Discussion

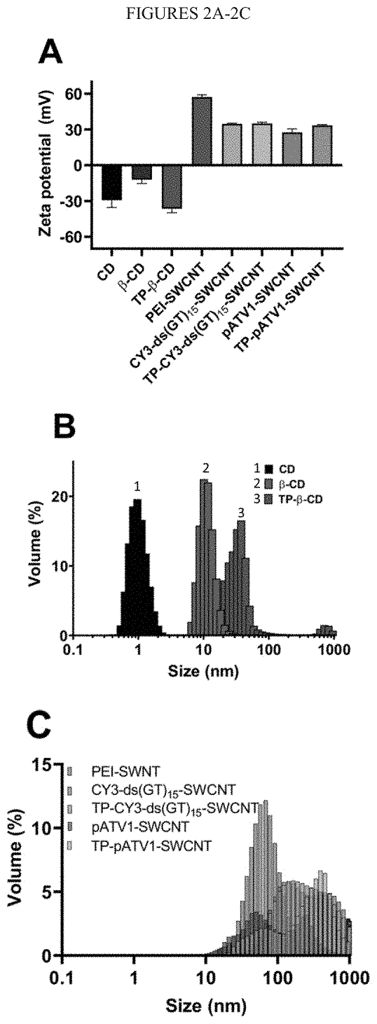

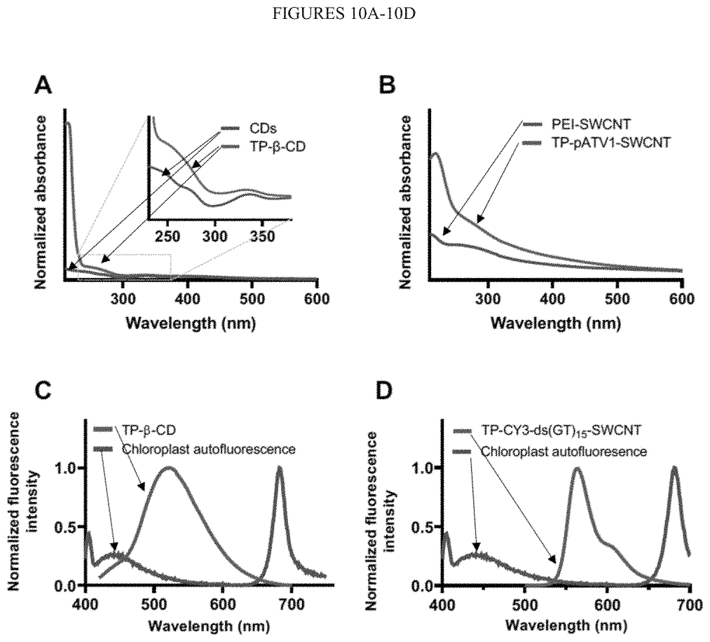

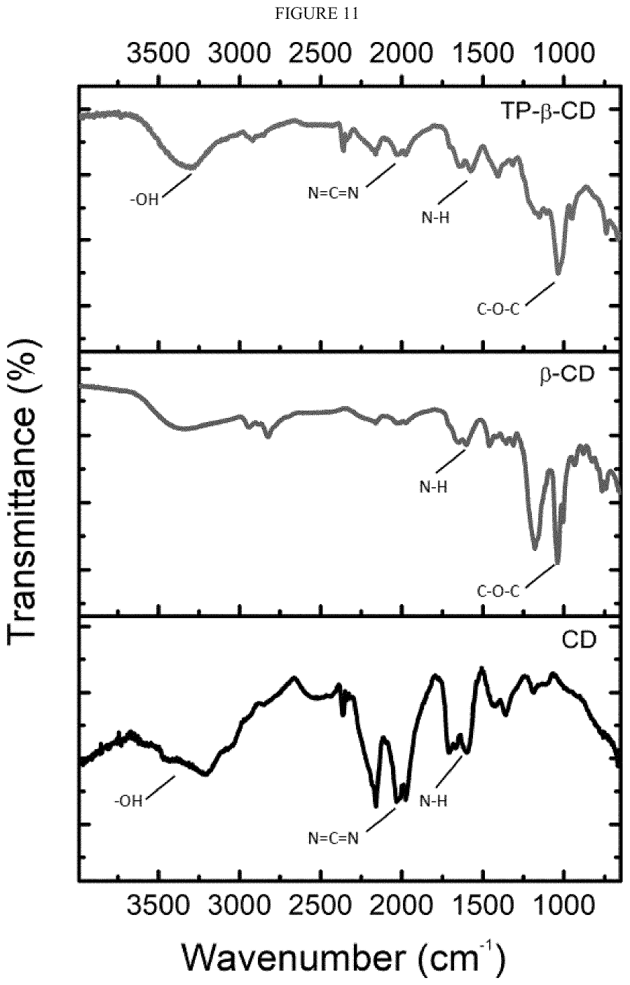

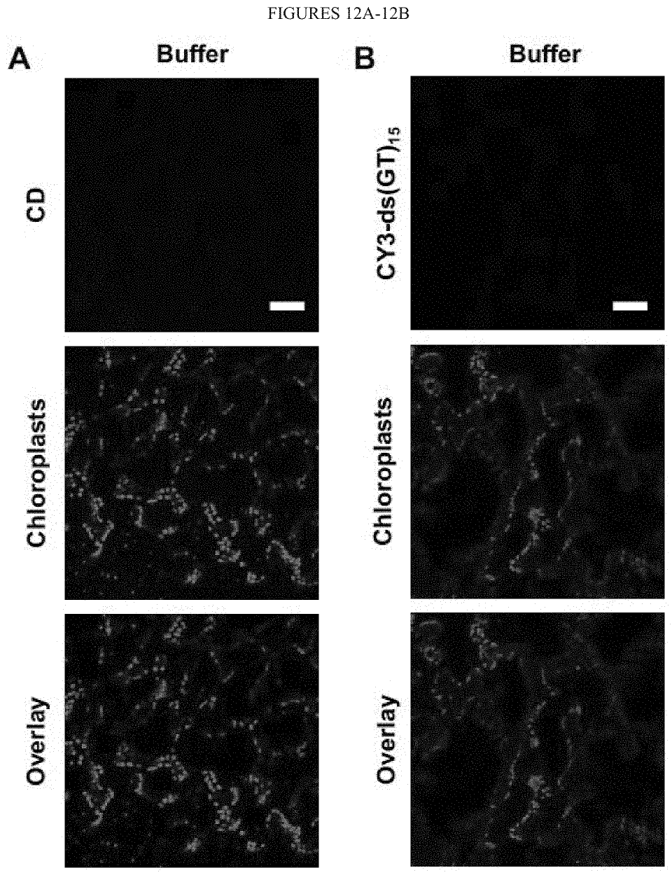

Targeted Carbon Nanomaterials for Chemical and Gene Delivery. The UV-vis absorbance spectrum of targeted CDs indicated characteristic absorption peaks at 272 nm for π-π transition of C═C bonds and 335 nm for n-π* transition of C═O or C═C bonds, respectively (FIG. 10).⁴⁴⁻⁴⁶ Nontargeted PEI coated single-walled carbon nanotubes (PEI-SWCNTs) showed absorption peak shoulders around 260 nm due to the PEI polymer (FIG. 10).⁴⁷ Both CD and TP-pATV1-SWCNT absorbance spectra broadening at 215-350 nm range are attributed to the surface functionalization with biomolecules such as peptides, DNA or β-cyclodextrin.^{48,49,46} The ζ potential of β-CD (−12.1±3.2 mV) decreased after functionalization with the chloroplast targeting peptide in TP-β-CD (−36.4±3.4 mV) (10 mM TES buffer, pH 7.0) (FIG. 2A). The chloroplast targeting peptide for CD (MASSMLSSATMVGGC (SEQ ID NO:5)) has a neutral charge (−0.1 mV) that upon covalent bonding to positively charged NH₂ groups in β-cyclodextrins through a NHS-PEG₄-MAL linker results in the decrease in ζ potential for TP-β-CD. The ζ potential for PEI-SWCNT decreased from 57.3±1.9 mV to 33.4±0.76 mV for targeting peptide coated TP-pATV1-SWCNT (10 mM TES and 0.1 mM NaCl, pH 7.0) (FIG. 2A). The electrostatic interactions between the negatively charged plasmid DNA (pATV1) and the positively charged PEI on the SWCNT surface decrease the ζ potential of TP-pATV1-SWCNT. The CY3-ds(GT)₁₅-SWCNT and TP-CY3-ds(GT)₁₅-SWNT had similar ζ potentials of 34.6±0.6 and 35.1±0.9 mV, respectively (FIG. 2A). The hydrodynamic size for CY3-ds(GT)₁₅-SWCNT and TP-CY3-ds(GT)₁₅-SWCNT was also similar, 221.8±13.02 nm and 216.7±8.23 nm, respectively (FIG. 2C). Both targeted TP-β-CDs and TP-pATV1-SWCNTs have a highly negative or positive charge, respectively, that has been shown to promote uptake through chloroplast envelopes and plasma membranes in vitro²³ and leaf biosurfaces in vivo.¹⁸ The hydrodynamic size for β-CD measured by dynamic light scattering increased from 10.2±1.5 nm to 27.8±5.8 nm for TP-β-CD (FIG. 2B). Likewise, the average DLS size for the pATV1-SWCNT and TP-pATV1-SWCNT increased from 49.98±3.45 nm to 382.5±27.0 nm, respectively (FIG. 2C). The increase in DLS size is associated with the coating of pATV1-SWCNT with a modified chloroplast targeting peptide (30 residues,

(SEQ ID NO:4))
MASSMLSSATMVGGGGGGKHKHKHKHKHKH.

The Fourier-transform infrared spectroscopy (FTIR) analysis of CDs indicated characteristic bonds for O—H stretching vibrations at 3240 cm⁻¹, C≡C alkyne 2160 cm⁻¹, carboxamides N═C═N at 2010 cm⁻¹. The peaks near 1700 cm⁻¹ and 1650 cm⁻¹ were attributed to C═O conjugated aldehydes and N—H amine bonds^{33,50} (FIG. 11). The β-CDs exhibited significant characteristic peaks for asymmetric glycosidic vibration bonds (C—O—C) of β-cyclodextrins at 1040 cm⁻¹ (FIG. 11).^{33,51} The FTIR of targeted TP-β-CDs exhibited peaks at O—H stretching vibrations at 3240 cm⁻¹, asymmetric glycosidic vibration (C—O—C) at 1050 cm⁻¹, and bands typical of type I amide bonds at 1610 cm⁻¹, supporting the successful conjugation of β-cyclodextrin and targeting peptides on the CD surface (FIG. 11). The CDs exhibited a fluorescence emission peak at 511 nm, and the SWCNTs coated in CY3-ds(GT)₁₅ (SEQ ID NO: 6) DNA showed a fluorescence emission peak at 564 nm. These nanomaterial fluorescence peaks of emission allowed tracking inside plant cells with minimum overlap with chloroplast autofluorescence background (FIG. 10).

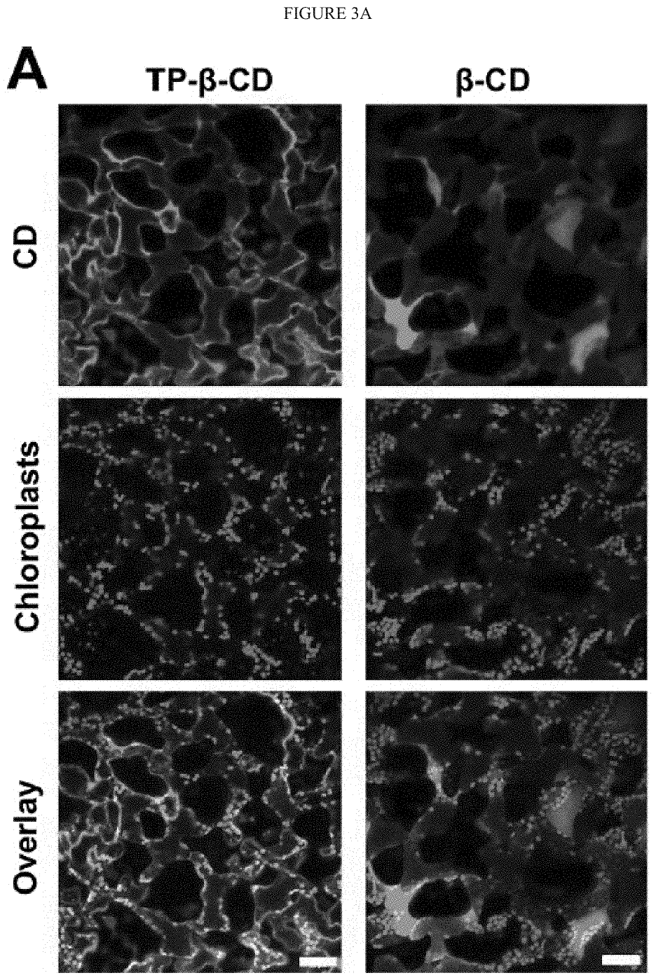

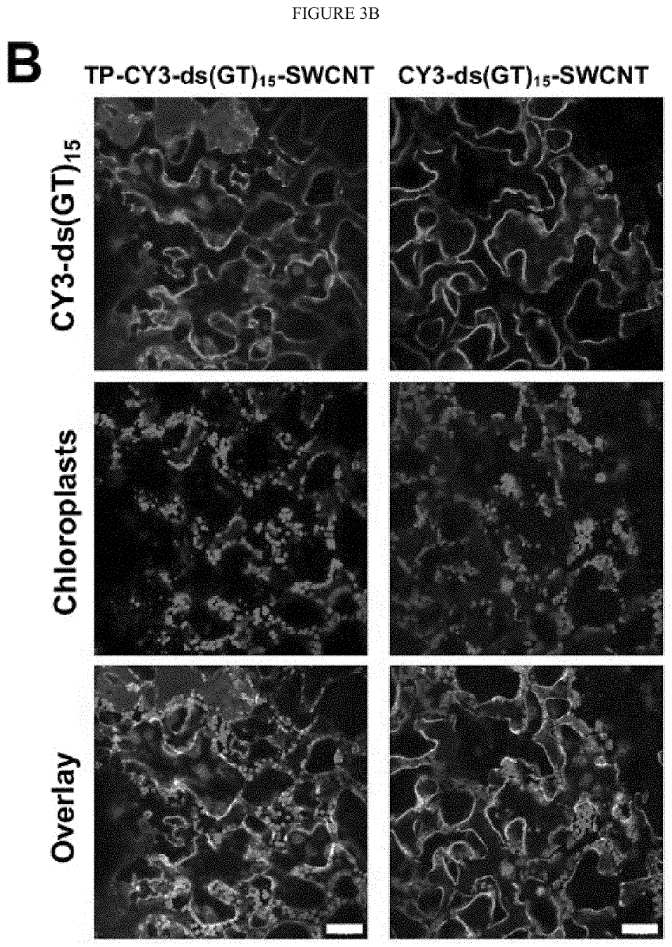

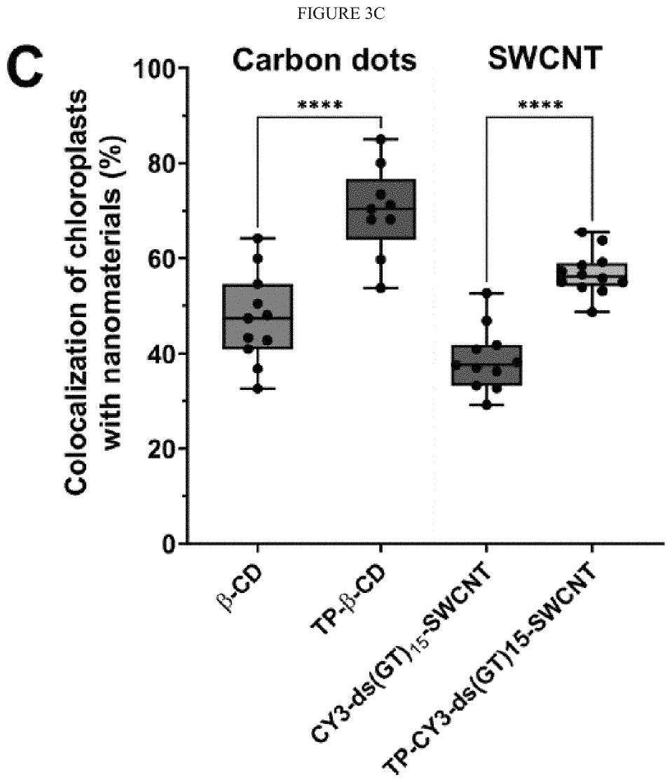

In Vivo Imaging of Chloroplast Targeted Nanomaterials. Confocal fluorescence microscopy imaging was used to determine the colocalization between chloroplasts in Arabidopsis leaves and both targeted and nontargeted β-CDs and CY3-ds(GT)₁₅ (SEQ ID NO: 6) DNA coated SWCNT (FIG. 3A,B). Nanomaterials were foliar sprayed on whole plants in a formulation containing 0.1% Silwet (v/v) (Table 1). Control confocal images of leaves treated with only 10 mM TES buffer have no background autofluorescence in CD and CY3-ds(GT)₁₅ (SEQ ID NO: 6) DNA emission channels (FIG. 12). The level of colocalization of fluorescent emission from nanocarriers with chloroplasts was determined by Manders' coefficient analysis (COLOC2, ImageJ) (FIG. 3C). The localization of targeted nanomaterials (TP-β-CD and TP-CY3-ds(GT)₁₅-SWCNT) with chloroplasts in leaf mesophyll cells was higher compared to nontargeted materials lacking the targeting peptide (β-CD and CY3-ds(GT)₁₅-SWCNT). The colocalization rates for TP-β-CDs with chloroplasts significantly increased to 70.0±9.46% from 47.4±9.57% levels for β-CDs and similarly to 56.9±4.58% for TP-CY3-ds(GT)₁₅-SWCNT from 38.7±6.69% for CY3-ds(GT)₁₅-SWCNT (p<0.0001) (FIG. 3C). Previously, we reported an in vivo increase in colocalization of chloroplasts with heavy-metal-based quantum dots functionalized with chloroplast targeting biorecognition peptides,¹⁶ indicating the robustness of this approach for a variety of targeted nanomaterials. The use of foliar delivery of nanomaterials onto leaves provides a facile, efficient, and scalable method to interface nanomaterials with crops.^{21,52} Foliar chemical delivery approaches mediated by nanomaterials in plants can improve the efficacy of fertilizer, pesticides, and other agrochemicals for enhancing plant growth.

The colocalization of TP-β-CDs and TP-CY3-ds(GT)₁₅-SWCNTs with chloroplast inside leaf mesophyll cells indicates that these nanomaterials are able to translocate across the leaf surface and through plant cell barriers including the cell wall, plasma membrane, and chloroplast envelopes. The nanomaterial uptake pathway is likely micron-sized stomatal pores through which the translocation of CDs in crop leaves might occur.¹⁸ Although, stomatal density is lower on the upper (adaxial) leaf surface than on the lower (abaxial) side in Arabidopsis as in most plants, the high levels of colocalization of nanomaterials with chloroplasts indicate that this does not constitute a limitation for the translocation of the nanomaterials through plant cell barriers. The larger size of the TP-β-CDs and TP-CY3-ds(GT)₁₅-SWCNTs compared to their untargeted counterparts does not decrease their ability to translocate across plant cell barriers and reach the chloroplast target. To the contrary, the coating with targeting peptide biorecognition motifs improved the delivery efficiency to chloroplasts. These results indicate that more research is needed to understand how biomolecule coatings affect the permeability of nanomaterials through plant cell walls reported to act as size exclusion barriers for nanoparticles.⁵³ The ζ potential of TP-β-CDs and TP-CY3-ds(GT)₁₅-SWCNTs was higher in magnitude than 30 mV, which may allow delivery of nanomaterials into chloroplasts in vitro.^{18,22,23} Previous studies have reported that highly charged CDs and nanotubes may move across leaf cell barriers into chloroplasts in vivo.^{18,54}

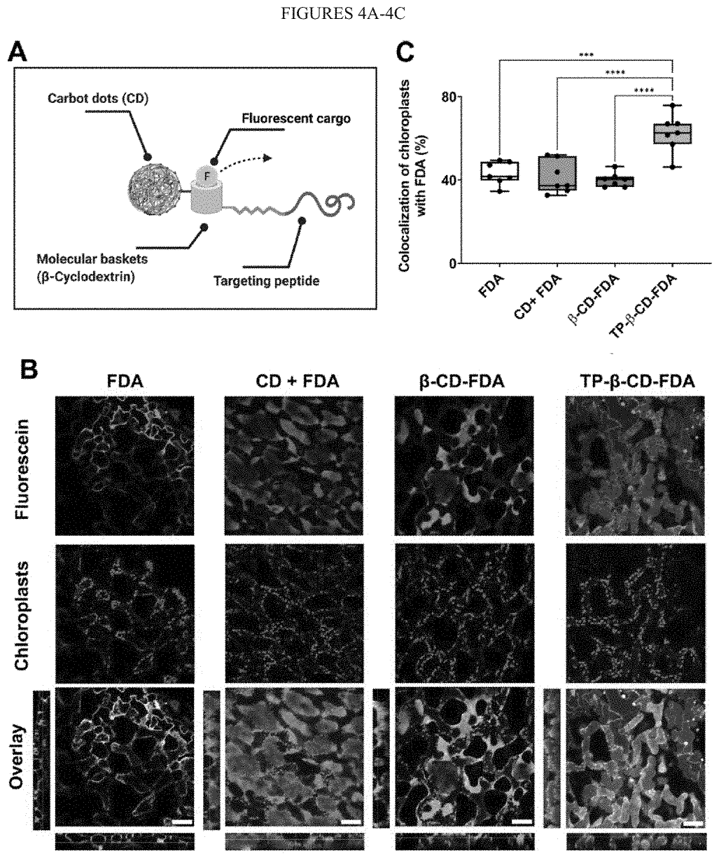

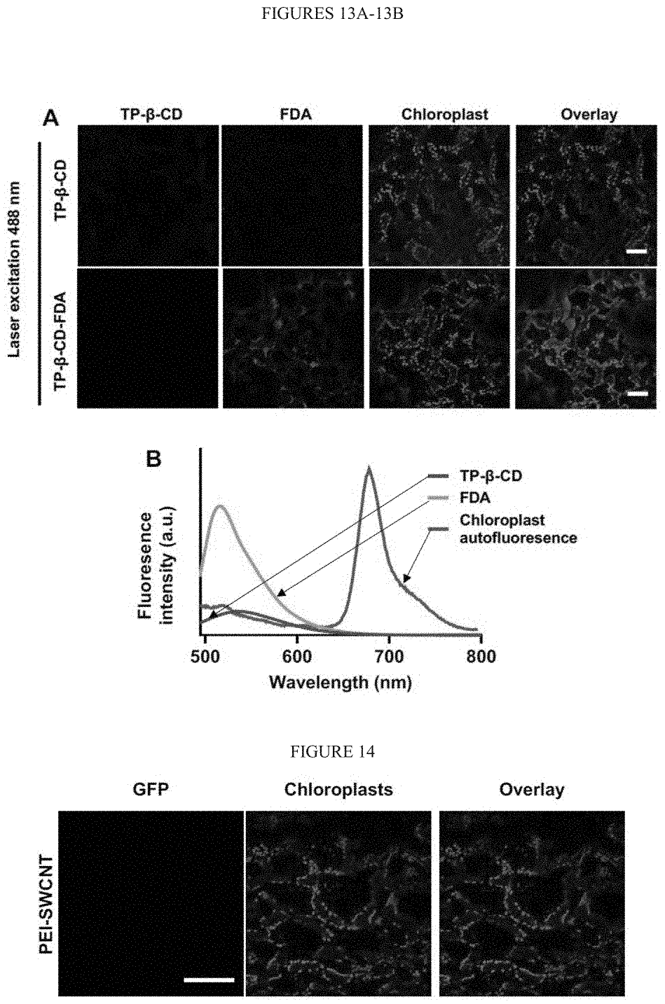

Chemical Cargo and Plasmid DNA and Delivery to Chloroplasts Mediated by Targeted Nanomaterials. To assess the delivery of chemical cargoes to chloroplasts mediated by TP-β-CD nanocarriers, we loaded β-cyclodextrins with the fluorescent dye 6-carboxyfluorescein (FDA) (FIG. 4A). We imaged the localization of FDA within leaf mesophyll tissues by confocal microscopy and quantified the localization of this fluorescence dye with chloroplasts autofluorescence (FIG. 4B,C). Plant leaves treated with TP-β-CD-FDA or TP-β-CD (Table 1) were analyzed for the fluorescence emission crosstalk with confocal microscopy under laser excitation with a 488 nm laser (FIG. 13A). FDA dye emission spectra exhibited a much stronger fluorescence signal compared to TP-β-CDs at 488 nm excitation allowing detection of loaded cargos to chloroplasts with a minimal crosstalk from TP-β-CDs emission (FIG. 13B). The FDA alone localized near the plasma membrane. When added together with CDs or β-CDs, the FDA was observed both in the intracellular and extracellular space. As shown in 2D plane projections in the XZ and YZ axis from orthogonal Z-stack images (FIG. 4B), the FDA delivered by CD+FDA and β-CD-FDA localized with a fraction of chloroplasts at a colocalization rate of 44.5±6.4 and 47.0±9.6%, respectively. In contrast, when FDA was delivered by TP-β-CDs, most of the FDA fluorescence signal was detected inside leaf mesophyll cells and highly colocalized with chloroplasts (70.0±9.5%) (FIG. 4B). Using the 2D plane Z-projections, we confirmed this distribution analysis of FDA in the leaf mesophyll and the localization with chloroplasts (FIG. 4B). Despite that the TP-β-CD DLS size is larger than their nontargeted counterparts (FIG. 2B), these nanocarriers more efficiently deliver chemical cargo to chloroplasts, indicating that their high ζ potential (>30 mV) and targeting peptide coating may play a more important role in determining their translocation efficiency through leaf mesophyll cells. Although targeted TP-β-CDs improved the delivery of FDA to chloroplasts in leaf mesophyll cells, the FDA fluorescence signal was localized throughout the entire leaf mesophyll cells. This might indicate that the FDA cargo was also released inside the cell cytosol before reaching chloroplast target organelles. The β-cyclodextrin surface chemistry can be modified for controlled release of cargoes (i.e., pH),⁵⁵ providing a pathway to more efficiently deliver the chemical cargo to the intended target. Colocalization rates of FDA fluorescence with chloroplasts were determined using Mander's coefficient analysis (COLOC2, ImageJ). The colocalization rates of TP-β-CDs loaded with FDA were compared to FDA dye only, core CDs mixed with FDA, and β-CDs loaded with FDA (FIG. 4C). The percent localization of FDA delivered by TP-β-CDs (62.5±9.22%) was significantly higher compared to nontargeted β-CDs (40.0%±3.42) (***p<0.0002, ****p<0.0001) (FIG. 4C). Targeted delivery to chloroplasts by TP-β-CDs provides a traceable fluorescent nanotechnology-based tool with biocompatibility^{18,56} and degradability^{32,57,58} for agrochemical delivery in plants with improved subcellular delivery precision and cell uptake efficiency. The development of targeted chemical delivery approaches in plants can aid in improving the efficacy of agrochemical delivery while minimizing unintended pollution in the environment.^{21,59}

We investigated the targeted delivery of plasmid DNA and expression in chloroplasts of Arabidopsis leaves mediated by biorecognition of SWCNTs. We used pATV1, a dicistronic plasmid encoding for both a GFP and antibiotic resistance genes regulated by a chloroplast promoter that is codon-optimized for specific expression in chloroplasts (FIG. 5A).⁴³ The pATV1-SWCNTs were coated with a modified chloroplast targeting peptide MASSMLS SATMVGGGGGGKHKHKHKHKHKH (SEQ ID NO:4) that contains a 12 lysine and histidine residues (KH₆) tail (SEQ ID NO: 2). The KH₆ peptide tail (SEQ ID NO: 2) enables the electrostatic binding of the chloroplast targeting peptide to the negatively charged plasmid DNA in pATV1-SWCNTs, and the G₆ spacer (SEQ ID NO: 3) enhances the exposure of the biorecognition motif to chloroplast membrane receptors.⁶⁰⁻⁶² The GFP fluorescence was imaged by confocal microscopy in leaf mesophyll cells (FIG. 5B). A 3D rendering of GFP expression and chloroplasts autofluorescence indicated high levels of GFP in these organelles in selected leaf mesophyll cells treated with targeted TP-pATV1-SWCNTs (FIG. 5C). Confocal analysis of GFP fluorescence emission in plant leaves treated with TP-pATV1-SWCNTs exhibited a more robust GFP signal and higher colocalization within chloroplast in vivo (56.7±6.0%) than nontargeted pATV1-SWCNTs (37.0±6.3%) (FIG. 5D). Despite that TP-pATV1-SWCNTs have a larger DLS size than their nontargeted counterparts and similar ζ potential (FIG. 2A,B), the targeting peptide coated nanocarriers more efficiently deliver DNA to chloroplasts, indicating that biorecognition mechanisms may exert a stronger influence on their translocation through leaf biosurfaces. No background fluorescence was detected in the GFP emission range when Arabidopsis plants were treated with only PEI coated SWCNTs (FIG. 14).

Expression analysis results indicated that the peak of mRNA transcription levels for GFP is reached after 5 days of exposure for either TP-pATV1-SWCNT or pATV1-SWCNT, which is followed by a decrease in GFP gene expression at day seven (FIG. 5E). Interestingly, despite higher localization of targeted TP-pATV1-SWCNTs with chloroplasts, both TP-pATV1-SWCNTs and pATV1-SWCNTs exhibited similar levels of GFP mRNA. This potentially indicates that functionalization of plasmid DNA coated SWCNTs with targeting peptides increases localization with chloroplasts, but it may lead to interference with the plasmid DNA expression by chloroplasts. An alternative explanation is that the targeted TP-pATV1-SWCNTs cause higher rupturing of the chloroplast membranes (as demonstrated in experiments below), thus decreasing overall gene expression in the chloroplasts. The RT-qPCR analysis of GFP mRNA expression was compared to the relative change in expression of the internal housekeeping gene Actin2 (AT3G18780).^{63,64} GFP expression was also confirmed by quantifying GFP protein levels with an ELISA assay of the total soluble proteins of leaf extracts. Maximum levels of GFP protein were detected in the total soluble proteins after 5 days of treating leaves with both TP-pATV1-SWCNT and pATV1-SWCNT, followed by a reduction in GFP levels at day 7 (FIG. 5F). This additional quantitative analysis mirrors the trend observed in RT-qPCR GFP gene expression (FIG. 5E) and provides an orthogonal line of evidence of GFP production by chloroplasts. SWCNTs functionalized with positively charged chitosan have been reported to deliver plasmid DNA to chloroplasts in plant leaves where gene expression was assessed by confocal fluorescence microscopy.¹⁹ Herein, we report that biorecognition-mediated delivery of plasmid DNA to chloroplasts by targeted SWCNT enables high levels of transient transgene expression using confocal microscopy, quantitative gene expression and protein level analysis.

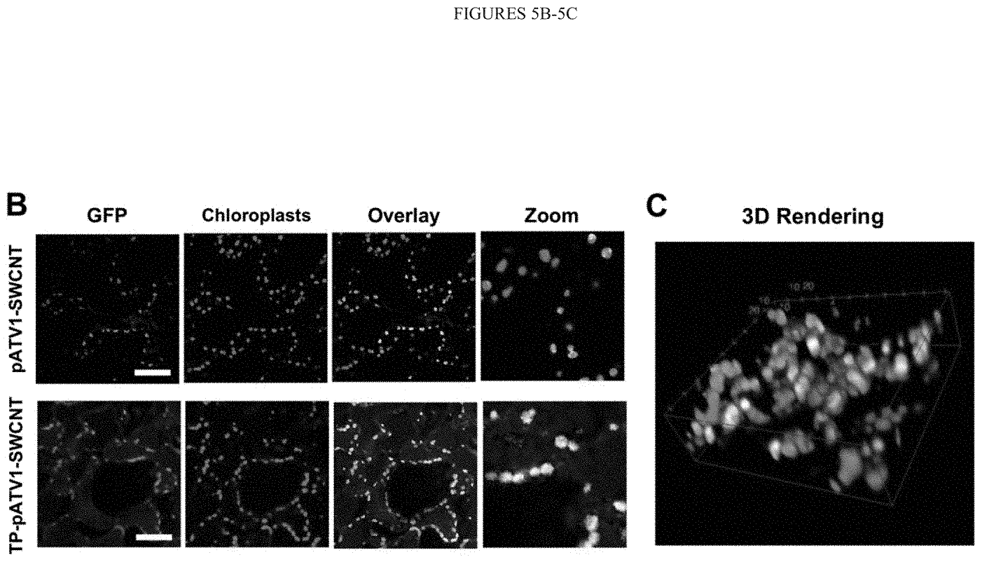

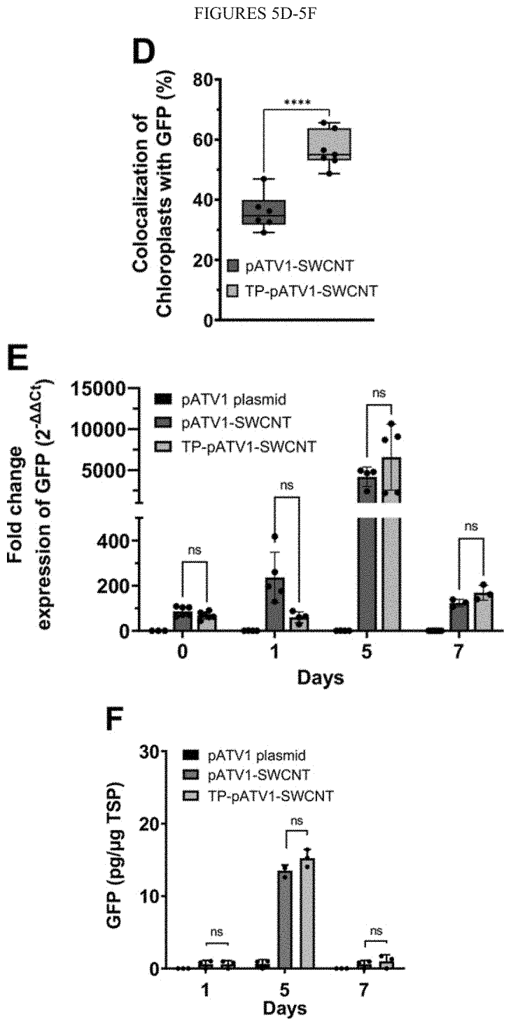

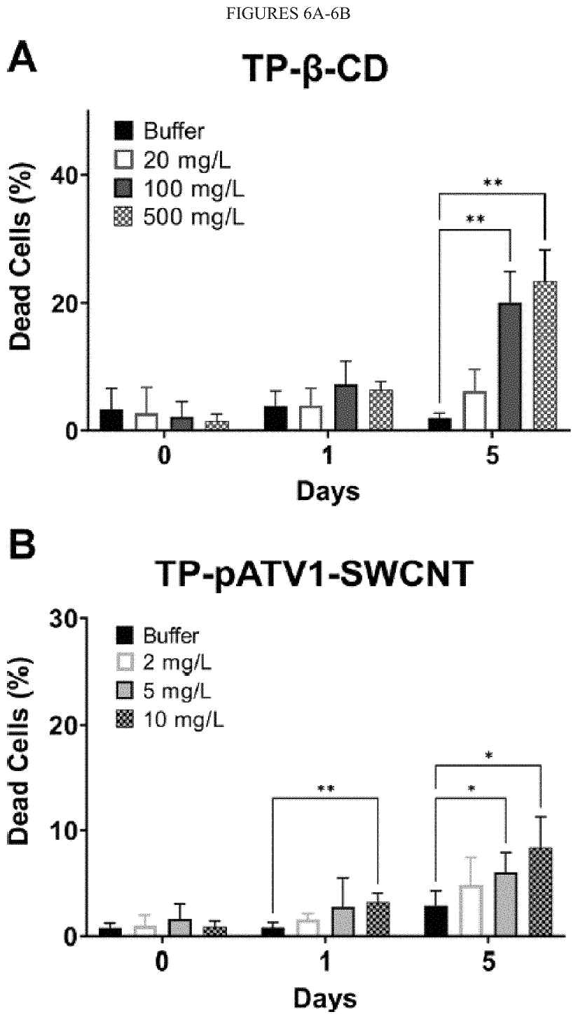

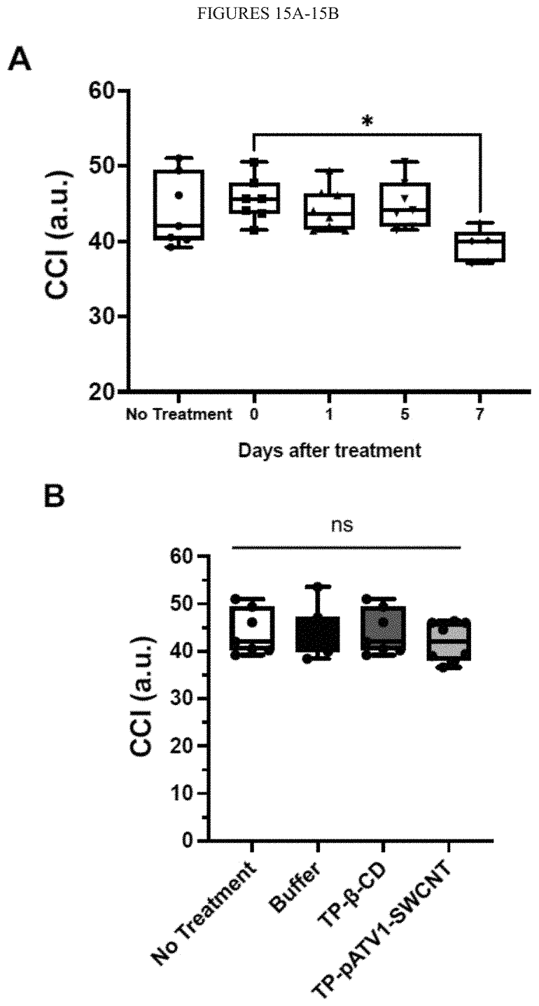

Impact of Targeted Nanomaterials on Plant Cell Viability. We investigated the viability of plant cells treated with increasing concentrations of targeted and nontargeted nanomaterials by measuring the percentage of dead plant cells. Arabidopsis leaf tissues were treated with targeted nanomaterials at 20, 100, 500 mg/L concentrations of TP-β-CDs or 2, 5, 10 mg/L of TP-pATV1-SWCNTs (FIG. 6, Table 1). Biocompatibility assays were performed up to 5 days of nanocarrier exposure before control Arabidopsis plants experienced the onset of leaf senescence marked by significant changes in chlorophyll levels (FIG. 15A). On day 7, significant differences in chlorophyll were detected in control Arabidopsis plants without nanomaterials. The percentage of intact cells at day 1 for TP-β-CDs (20 mg/L) and TP-pATV1- SWCNTs (2 mg/L) was biocompatible, resulting in no significant differences in plant dead cells relative to controls without nanoparticles over the same experimental time frame (FIG. 6A,B). However, the TP-β-CDs exhibited significantly higher percentages of dead cells on day 5 of exposure at concentrations of 100 mg/L (20.0±4.8%) and 500 mg/L (23.4±4.8%) compared to controls (2.0±0.7%) (FIG. 6A). Similarly, the TP-pATV1-SWCNT treated leaves showed an increase in cell death on day 5 at concentrations of 5 mg/L (6.0±1.9%) and 10 mg/L (8.4±2.9%) relative to controls (2.9±1.4%) (FIG. 6B). Therefore, subsequent experiments assessing the impact of targeted nanocarriers on plant cell and molecular biology were focused on biocompatible concentrations of 20 mg/L for TP-β-CDs and 2 mg/L for TP-pATV1-SWCNTs.

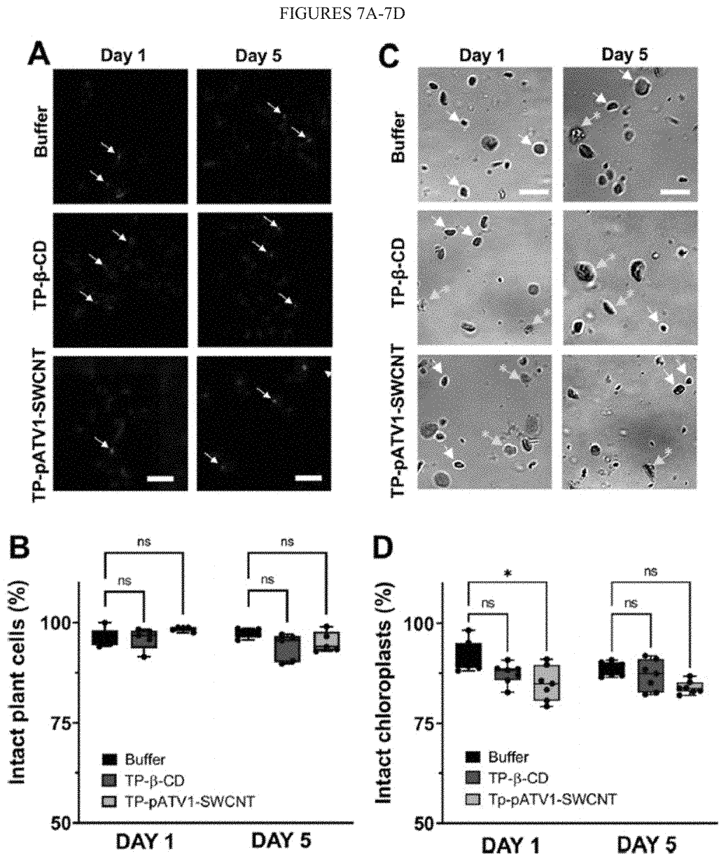

Interactions of Targeted Carbon Nanostructures with Plant Cell and Chloroplast Membranes. Damage to plant lipid membranes may cause ion and molecule permeability changes across the membrane, interruption of metabolic processes, intracellular signaling, and trafficking of biomolecules.^{65,66} The application of targeted nanostructures with high charge allows penetration of plant cell barriers and localization inside organelles that could cause disruption in lipid membrane integrity. Plant cell membrane intactness in leaves treated with TP-β-CDs or TP-pATV1-SWCNTs (Table 1) was assessed by staining dead cells with propidium iodide (PI) dye followed by imaging under confocal microscopy (FIG. 7A). PI is a nonpermeable dye that only crosses the cell membranes when they are damaged. The overall percentage of intact cells without PI stained nuclei was calculated relative to the total number of cells (FIG. 7B). Targeted nanomaterials did not have a significant impact on plant cell membrane intactness. Both TP-β-CDs and TP-pATV1-SWCNTs maintained more than 93% of plant cells with intact membranes after 1 and 5 days of exposure, similar to controls without nanoparticles (FIG. 7B). In addition, we performed identification of intact chloroplasts by differential interference contrast (DIC) microscopy as reported previously with some modifications.^{67,68} Intact isolated chloroplasts observed by DIC microscopy have a highly reflective and continuous outer envelope, whereas damaged chloroplasts have a broken envelope with opaque and granular appearance (FIG. 7C). The TP-β-CDs did not affect chloroplast membrane damage during the study period (FIG. 7D). In contrast, the TP- pATV1-SWCNTs induced a significant decrease in chloroplast intactness after 1 day of exposure (FIG. 7D). As proposed by the LEEP model, lipid exchange between SWCNT and chloroplast envelopes as the nanomaterials enter these organelles^{22,23} could explain the temporary decrease in chloroplast membrane intactness. Our results indicate that high aspect ratio SWCNTs, but not CDs, could result in disruption of plant lipid membrane structures.

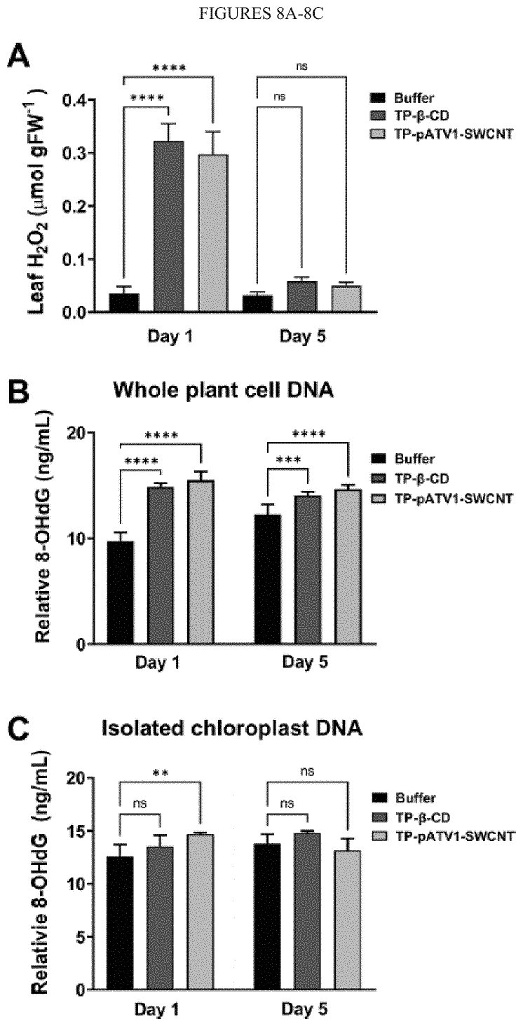

Transient Increase in Leaf H₂O₂ Content after Nanomaterial Exposure. The impact of nanomaterials for targeted delivery of chemical cargoes and DNA on chloroplasts' reactive oxygen species (ROS) levels has not been explored. Chloroplasts are main sites for ROS generation.^{65,69} We used a quantitative peroxide assay to monitor H₂O₂ content in leaves after treatment with targeted nanomaterials (FIG. 8A) (Table 1). The TP-β-CDs and TP-pATV1-SWCNTs increased leaf H₂O₂ levels to 0.3225±0.0190 and 0.2970±0.0341 μmol gFW-1, respectively, after 1 day of exposure, whereas control plants exhibited 0.0347±0.0088 μmol gFW-1 values. The observed 10-fold increase in leaf H₂O₂ was within normal H₂O₂ levels reported for nonstressed land plants (<5 μmol gFW-1).⁷⁰⁻⁷⁴ However, H₂O₂ levels can vary significantly within plant species and even organs within plants.^{70,71} Leaf H₂O₂ decreased to levels similar to controls (0.0320±0.0021 μmol gFW-1) after 5 days of exposure to TP-β-CDs (0.0583±0.0033 μmol gFW-1) and TP-pATV1-SWCNTs (0.0496±0.0029 μmol gFW-1) (FIG. 8A). Although plants have mechanisms to catalytically scavenge ROS, the transient increase in the levels of leaf H₂O₂ could cause damage to DNA, chlorophyll pigments, and photosynthetic proteins.^{73,75-78}

Oxidative Damage to Cell and Chloroplast DNA by targeted nanomaterials. To gain insight into the impact of targeted nanomaterials on plant cell and chloroplast genomes, we measured the relative 8-hydroxy-2′-deoxyguanosine (8-OHdG) levels in whole plant cell DNA and chloroplast DNA upon exposure to targeted nanomaterials (Table 1). Increased in ROS levels may result in DNA damage and the production of 8-hydroxydeoxyguanosine (8-OHdG), a ubiquitous biomarker in the guanine of nucleic acids.^{79,80} Significantly higher levels of 8-OHdG biomarkers in whole plant cell DNA were observed after 1 day of leaf exposure to TP-β-CD (14.9±0.3 ng/mL) and TP-pATV1-SWCNT (15.5±0.8 ng/mL) relative to controls without nanoparticles (9.7±0.8 ng/mL) (FIG. 8B). On day 5, the levels of 8-OHdG biomarkers remained slightly more elevated in both treatments with TP-β-CDs (14.9±0.3 ng/mL) and TP-pATV1-SWCNTs (14.6±0.4 ng/mL) relative to controls without nanoparticles (12.3±0.9 ng/mL) (FIG. 8B). In isolated chloroplast DNA samples from leaves treated with TP-pATV1-SWCNT, we also observed initial DNA oxidative damage on day 1 of exposure (14.7±0.2 ng/mL), but after 5 days of exposure to the targeted nanomaterials, the 8-OHdG levels were similar to controls (TP-pATV1-SWCNT 13.2±1.1 ng/mL; control 13.8±0.9 ng/mL) (FIG. 8C). In contrast, in leaves treated with TP-β-CDs, the levels of chloroplast 8OH-dG biomarkers 1 day after exposure (13.5±1.0 ng/mL) were similar to that of controls without nanoparticles (12.6±1.1 ng/mL) (FIG. 8C). Accumulation of H₂O₂ may inhibit DNA repair mechanisms, allowing lesions and DNA damage in the plant nuclear genome to accumulate.^{73,81} In contrast, plastid genomes such as those of chloroplasts are highly dynamic and contain hundreds of copies relative to the single nuclear genome in plant cells. If damaged plastid DNA exceeds the capacity of repairing mechanisms, the damaged DNA is fragmented and degraded, and new DNA is replicated.^{82,83}

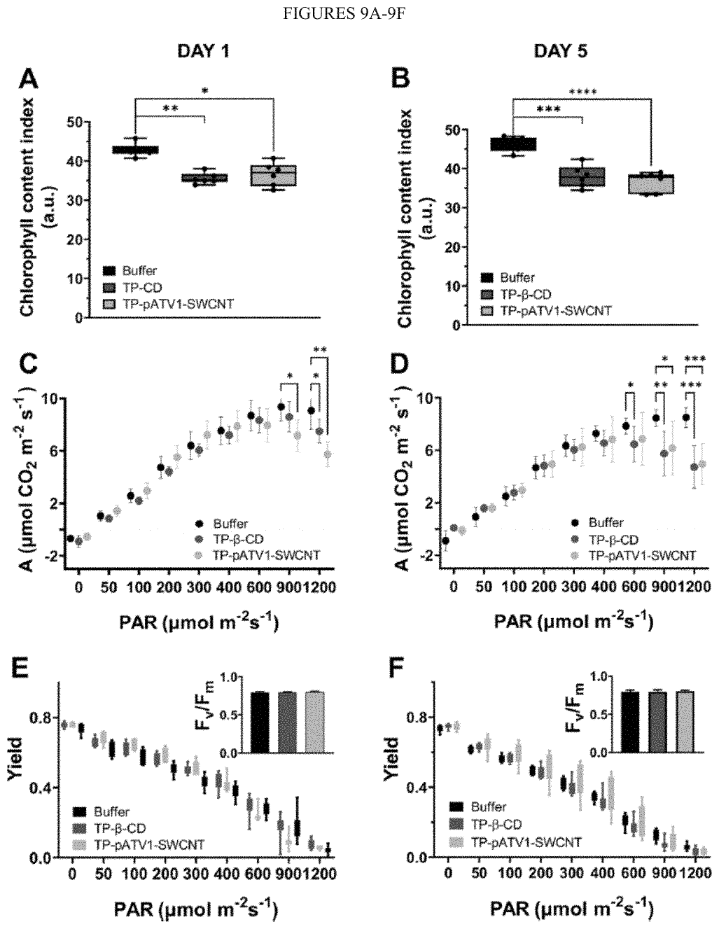

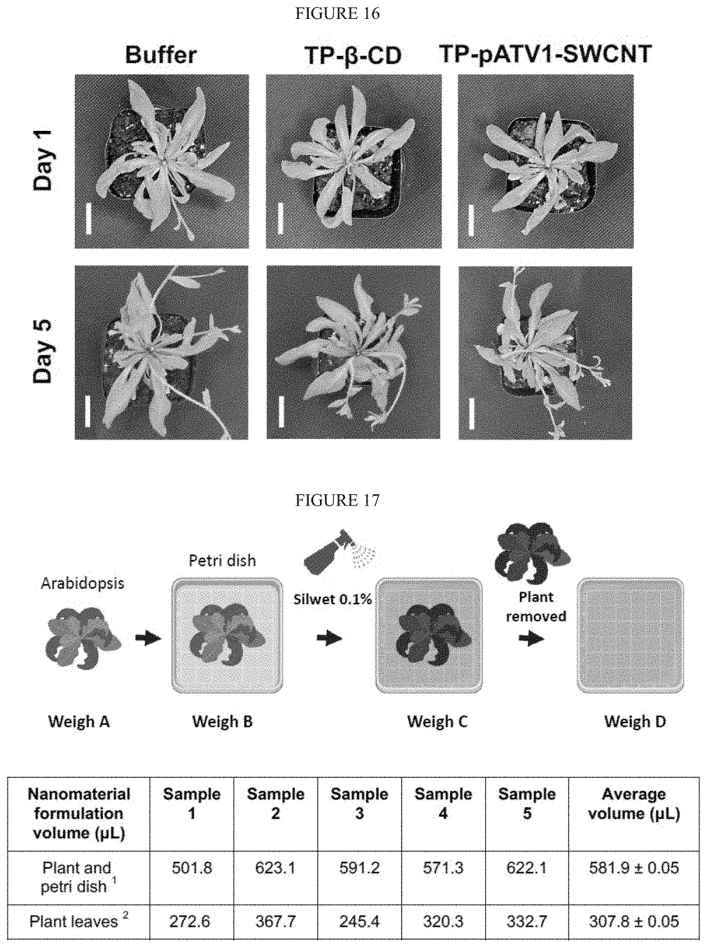

Effects of Targeted Nanomaterials on the Light and Carbon Reactions of Photosynthesis. ROS can damage chlorophyll pigments and reduce their biosynthesis in chloroplasts.⁸⁴ Chlorophyll is a marker for plant health status.^{85,86} We determined the impact of targeted nanomaterials (Table 1) on chlorophyll content index (CCI) of Arabidopsis leaf tissues. After 1 day of exposure to targeted nanomaterials, the CCI of leaves interfaced with TP-β-CDs (35.5±1.4) and TP-pATV1s (36.6±3.0) was significantly lower than controls without nanoparticles (42.8±1.7) (FIG. 9A,B). A similar trend was observed in leaf CCI values after 5 days of exposure to TP-β-CDs (38.0±2.8) and TP-pATV-SWCNTs (36.6±2.5) and controls (46.3±2.0) (FIG. 9A,B). We verified that there is no interference in CCI measurements with absorbance of the applied targeted nanomaterials by measuring leaf CCI before and after treatment with nanomaterials (FIG. 15B). However, there were minimal signs of localized chlorosis or necrosis on plant leaves after treatment with targeted nanomaterials during this time frame (FIG. 16). Previously, we reported no significant changes in chlorophyll content after the treatment with carboxylated, aminated, and amphiphilic CD at concentrations from 500 to 5000 mg/L18 in crop plants (maize and cotton), indicating either a higher tolerance of crop plants to CDs or higher impact of targeted TP-β-CDs to chloroplasts on leaf health. The biocompatible concentration of nontargeted ss-DNA coated semiconducting SWCNTs in Arabidopsis plants (5 mg/L)⁸⁷ was similar to that for TP-pATV1-SWCNTs (2 mg/L) used in this study for targeted delivery of plasmid DNA. However, the TP-pATV1-SWCNT induced a decline in leaf chlorophyll content after 5 days of exposure to the nanomaterials. Together, our results indicate that targeted delivery of TP-β-CDs and TP-pATV1-SWCNTs to chloroplasts in Arabidopsis plants can lead to the reduction in leaf chlorophyll content and that this effect on chloroplast pigments might be associated with a transient increase in ROS generation in leaves described above.

To assess the impact of TP-β-CD and TP-pATV1-SWCNTs on leaf photosynthesis, we measured carbon assimilation rates at varying photosynthetic active radiation levels (PAR). The photosynthesis light response curves provided information on the maximum leaf photosynthetic capacity (Aₘₐₓ) and photosystem II (PSII) quantum yield. The TP-β-CDs and TP-pATV1-SWCNTs did not influence carbon assimilation rates in the photosynthesis light-limited region (<400 μmol m⁻² s₋₁ of photosynthetic active radiation, PAR) at day 1 and at day 5 (FIG. 9C,D). However, we observed a reduction in Amax in the carboxylation limited region (>400 μmol m−2 s−1 PAR) at day 1 and day 5 relative to controls without nanoparticles (FIG. 9C,D). Nanomaterials with high surface charge have been reported to form protein coronas in organisms.⁸⁸⁻⁹¹ The localization of nanomaterials in chloroplasts could result in photosynthetic protein adsorption onto the surface of the nanomaterials. The nanomaterial interactions with enzymes and substrates of the carbon reactions of photosynthesis may be responsible for the decline in maximum photosynthetic capacity. In contrast, the quantum yield of PSII was not impacted by targeted nanomaterials within a wide range of PAR levels from 1 one to 5 (FIG. 9E,F). The maximum quantum yield of photosystem II (Fᵥ/Fₘ) in dark-adapted leaves for controls (0.79±0.02) (FIG. 9E,F, inset) was similar to that of targeted nanomaterial treated plants at day 1 (0.79±0.01, 0.80±0.02) and at day 5 (0.79±0.03, 0.80±0.01) for TP-β-CDs and TP-pATV1-SWCNTs, respectively (FIG. 9 E,F, inset). The Fᵥ/Fₘ is a robust indicator of the maximum quantum yield of PSII chemistry.⁹² A Fᵥ/Fₘ value in the range of 0.79-0.84 is optimal for many plant species, with lowered values indicating plant stress.⁹³ Together these results indicate that targeted nanomaterials do not impact the light-dependent reactions of photosynthesis nor damage the photosystems or the chloroplast electron transport chain. However, the nanomaterial interactions with carboxylation reaction biomolecules may limit the leaf photosynthetic capacity.

### Conclusions

We developed targeted carbon-based nanomaterials that deliver chemical cargoes (TP-β-CDs) and plasmid DNA (TP-pATV1-SWCNTs) to chloroplasts by plant biorecognition approaches. The application of targeted nanomaterials functionalized with guiding peptides as tools for plant bioengineering and precision agriculture may rely on the understanding of their impact on plant function. Cell viability assays of plants treated with TP-β-CDs (20 mg/L) and TP-pATV1- SWCNTs (2 mg/L) indicated no significant differences in the percentage of dead cells compared to control plants after 5 days of exposure. The targeted nanomaterials did not affect cell membrane intactness. However, TP-pATV1-SWCNT induced a temporary disruption of isolated chloroplast envelopes where chloroplast guiding peptides are recognized by membrane translocon channels.^{60,94,95} Because chloroplasts lack endocytosis-dependent mechanisms, nanoparticle uptake has been proposed to occur by disruption of the organelle envelopes followed by self-rehealing of the lipid bilayers.^{22,23} Exposure of plant leaf cells to targeted nanomaterials also induced a 10-fold transient increase in H₂O₂ levels relative to no nanoparticle controls. However, the leaf H₂O₂ content was within levels reported for healthy land plants.⁷⁰⁻⁷⁴ Elevated H₂O₂ concentrations can lead to damage of biomolecules such as DNA, lipids, and proteins^{81,96,97} and inhibit photosynthesis carboxylation rates in Arabidopsis thaliana plants.^{70,98,99} We observed a 2-fold increase in oxidative damage to whole plant cell DNA after exposure with TP-β-CDs and TP-pATV1-SWCNTs. In contrast, isolated chloroplast DNA was not affected by TP-β-CDs, while TP-pATV1-SWCNTs induced transient oxidative damage. The chloroplasts genome is highly dynamic, and self-repairing mechanisms^{82,83} could allow for rapid repair of damaged DNA or production of new DNA. A reduction in leaf chlorophyll content index levels after treatment with targeted nanomaterials could be attributed to increasing H₂O₂ levels.^{73,75-78} Despite this effect on chlorophyll pigments, photosystem II health and quantum yields across a wide range of light levels remained unchanged, indicating no impact on the light reactions of photosynthesis. However, a reduction in maximum photosynthetic capacity was observed in the carboxylation limited region of photosynthesis. Interactions between the nanomaterial surface and photosynthetic proteins involved in carbon fixation and assimilation could be responsible for the decline in photosynthetic capacity.

This study demonstrates that carbon nanomaterials engineered with targeting peptides increase the delivery efficiency of chemical and plasmid DNA cargoes into chloroplasts by topical application of the leaf surface. CDs with molecular baskets could act as biocompatible,²⁹⁻³¹ degradable,^{31,32} and traceable nanomaterials, made from renewable resources,^{18,33} for more precise delivery of active ingredients in crop plants, whereas pDNA-SWCNTs may enable transformation technologies for chloroplasts in plants in research facilities without the need of specialized equipment, tissue culture, or selection. Targeted nanomaterials overcome plant cell barriers including the cell wall, and lipid membranes, without mechanical aid, guided to chloroplasts by plant biorecognition. However, targeting of nanomaterials to the chloroplasts can induce transient increases in H₂O₂ levels that result in changes in whole leaf cell DNA and chlorophyll levels. Investigating scalable synthesis approaches, mechanisms of delivery, and safety of targeted carbon nanostructures will lead to sustainable nanotechnologies for improving agriculture. The results from this study provide insights for designing efficient and biocompatible nanomaterials for plant research, agriculture, and environmental applications.

### Methods

Plant Growth. Arabidopsis thaliana plants were grown in Adaptis 1000 growth chambers (Conviron) under the following environ-mental conditions: 200 μmol m⁻² s⁻¹ PAR, 24±1 and 21±1° C. day/night, 60% humidity, and 14/10 h (day/night) regime. All plants were grown in (2.5 in.×2.5 in.×3 in.) pots filled with soil containing 1% marathon and 1% osmocote. Plants were watered once every 3 days. Three-week-old Columbia ecotype (Col-0) Arabidopsis thaliana plants (seed stock source CS60000) in the prebolting stage were used for this study.

Covalent Modification of SWCNT with Polyethylenimine Polymer. Oxidized SWCNTs (>90%, 652490-250MG, Sigma- Aldrich) were functionalized using a branched polyethylenimine (PEI) polymer (10,000 MW, 9002-98-6, Alfa Aesar). The PEI positive charge of the SWCNT surface allows the electrostatic grafting of negatively charged DNA and other biomolecules. ^{19,24,100} First, 20 mg of oxidized SWCNTs were dispersed into 100 mL of ultrapurified water and pH adjusted to 12 with NaOH. The SWCNT solution was bath sonicated for 30 min at 80 kHz and 390 W power at room temperature. The resulting SWCNT solution was slowly poured into a PEI aqueous solution (2 mg/mL) while stirring. The mixture of PEI and SWCNTs was stirred for 30 min before placing in a heat-resistant Falcon tube and incubating for 16 h at 85° C. in a mechanical oven (Isotemp, Fisher scientific). The resulting PEI-SWCNT was cooled to room temperature and then resuspended in 15 mL of molecular biology grade water (catalog # 46000CV Corning) and bath sonicated. All bath sonication steps were conducted for 30 min at 80 Khz and 390 W power at room temperature unless stated otherwise. The resulting suspension was centrifuged for 10 min at 4500 rpm (Allegra X-3R, Beckman Coulter) at room temperature to remove large agglomerates. The PEI-SWCNT was further purified with molecular biology grade water (catalog # 46000CV Corning) to remove excess PEI polymer by washing five times through an MWCO 100 kD ultrafiltration microtube (VIVA SPIN 500, Sartorius). After each centrifugation step, PEI-SWCNTs were bath sonicated for 30 min to resuspend the nanomaterial pellet inside the VIVA SPIN 500 100 kD column after each washing. The PEI-SWCNT solution was centrifuged six times in a microcentrifuge tube at 13.2 RCF for 1 h to remove any remaining agglomerates. The lack of a dark pellet after centrifugation steps is an indicator of well-dispersed suspensions of SWCNT.

The resulting PEI-SWCNT suspension was characterized by measuring the absorbance spectra on a UV-vis absorbance spectrophotometer (UV-2600, Shimadzu). The quality of the SWCNT suspension was determined by analyzing absorbance peaks at 632 nm. This measurement was performed with triplicates. The concentration of the PEI-SWCNT was determined spectrophotometrically using the absorption value at 632 nm and utilizing the equation (absorbance at 632 nm/extinction coefficient of 0.036)=mg L⁻¹. The final concentration obtained after purification ranged from 18 to 30 mg/L. The nanoparticle ζ potential was measured using a Zetasizer (Nano ZS, Malvern Instruments) in samples suspended in 10 mM TES buffer pH 7.0, with 0.1 mM NaCl. The hydrodynamic size was measured using Zetasizer (Nano ZS, Malvern Instruments) in samples suspended in a 10 mM TES buffer at pH 7.

Electrostatic Grafting of pATV1 Plasmid on PEI-SWCNT. Loading of pATV1 plasmid onto the PEI-SWCNT was performed by electrostatic grafting, which allows molecules with negative charge to electrostatic interact with positively charged surfaces on the PEI-SWCNT. We used previously reported electrografting methods^{37,54} with some modifications. First, 0.01 mg of PEI-SWCNT with a net positive charge of 57.28±1.86 mV was suspended in 1 mL of 10 mM TES buffer (7365-44-8, Sigma-Aldrich) at pH 7. 0.01 mg of positively charged PEI-SWNTs was mixed with 0.02 mg of negatively charged pATV1 DNA plasmid in a 10 mM TES buffer (pH 7.0). The final ratio of PEI-SWCNT to pATV1 plasmid DNA was 1:2 (PEI-SWCNT:pDNA), and the final concentrations of PEI-SWCNT and pATV1 plasmid were respectively 10 mg/L and 20 mg/L in 10 mM TES buffer (pH 7). The PEI-SWCNT coated in pATV1 plasmids was denoted pATV1-SWCNT. The pATV1-SWCNT solution was then bath sonicated at room temperature for 15 min at 80 kHz with no further purification steps. The characterization of the pATV1-SWCNT was determined by measuring the change in UV absorbance spectra, hydrodynamic diameter, and ζ potential (Malvern ZetaSizer). Peptide Binding onto pATV1-SWCNTs. The pATV1-SWCNTs were functionalized with a chloroplast targeting peptide on their outer surface. The targeting peptide amino acid sequence was based on precursors of the conserved rubisco small subunit 1A (RbcS, genbank: OAP15425). Chloroplast targeting peptides have been utilized as a biorecognition motif that allows the import of nanomaterials and other nanoconjugates across the chloroplast membrane.^{16,101} To improve the delivery of pATV1-SWCNT into chloroplasts, we designed a chloroplast targeting peptide with a lysine histidine (KH₆) polypeptide tail (SEQ ID NO: 2) for enabling electrostatic binding to the plasmid DNA grafted onto the PEI-SWCNT (FIGS. 1 and 5A). The chloroplast targeting peptide motif also contains a flexible linker of six glycine residues (SEQ ID NO: 3) allowing increased stability in aqueous solutions and interaction with the biorecognition domains.^{16,103} Synthesis of the chloroplast TP (MASSMLSSATMVGGGGGGKHKHKHKHKHKH (SEQ ID NO:4)) was performed by Genscript. The TP was diluted in a stock solution of 10 mg/L in phosphate-buffered saline (PBS) solution (pH 7). A 0.1 mg of chloroplast targeting peptide was added to 1 mL of pATV1-SWCNT suspension (2 mg/L). The mass ratio of PEI-SWCNT:DNA:TP was 1:2:50. The resulting TP-pATV1-SWCNT nanostructure was incubated for 15 min while stirring at room temperature, followed by a bath sonication on ice for 15 min at 80 kHz, and no further purification steps, then suspended in a 10 mM TES buffer (pH 7) for subsequent experiments.

pATV1 GFP-Expressing Plasmid. The pATV1 plastid encoding GFP was obtained from Pal Maliga's lab (UCR-MTA19-0083, Rutgers University) and Giga prepped by Genewiz. The pATV1 vector (Genbank accession MF461355) carries a dicistronic operon, a Prrn16 promoter driving expression of the two open reading frames (ORF) encoding the aadA spectinomycin resistance gene, and the second ORF encodes the GFP protein (FIG. 5A).^{43,104} The homologous recombination site flanking construct could enable insertion into the inverted repeat region of the plastid genome. Polycistronic mRNAs are not translated on the eukaryotic type 80S ribosomes in the cytoplasm; thus, the accumulation of GFP fluorescence in chloroplasts of plants treated with SWCNT delivered pATV1 construct can be observed. The dicistronic nature of pATV1 and its chloroplast codon optimization allow specific assessment of GFP expression in chloroplast genomes of plants treated with pATV1-SWCNT.

Carbon Dots Synthesis. CDs were synthesized by a solid-state reaction^{18,33} using citric acid and urea. The CDs were further functionalized with a β-cyclodextrin molecular basket that enables chemical cargo loading into its cavity^{35,105} and a terminal chloroplast targeting peptide motif to import the nanomaterial containing a chemical cargo into chloroplasts.^{16,101} Briefly, 2.40 g of urea (40 mmol) (CAS # 57-13-6, 99.2%, Fisher Chemical), 1.92 g of citric acid (10 mmol) (CAS # 77-92-9, 99.7%, Fisher Chemical), and 1.35 mL of ammonium hydroxide (10 mmol) (NH₃·H₂O, 30-33%, Sigma-Aldrich) were added into 2 mL of molecular biology grade water. The mixture was dissolved, placed into a 50 mL beaker, and incubated in a mechanical oven at 180° C. for 1 h and 20 min. Following this reaction, the resulting CD suspension was allowed to cool down at ambient temperature, dissolved in water, and stirred for 1 h. This CD solution was bath sonicated for 15 min at 80 kHz with intermittent mixing by pipetting. Then, the solution was centrifuged at 4000 rpm for 15 min to remove large particles and aggregates. The supernatant was then filtered using a centrifugal filter 10 K Amicon filter (catalog # UFC901024, Amicon ultra, Merck Millipore) at 4500 rpm for 30 min to wash out unreacted precursors and small molecules. This step was repeated five times. Lastly, the solution was filtered through a 0.22 μm filter membrane (catalog # 229757, CELLTREAT Scientific Products) to obtain purified CD.

Cyclodextrin Functionalized Carbon Dots. The resulting core CDs are functionalized with mono-(6-ethanediamine-6-deoxy)-3-cyclodextrin (β-CD, Cavcon) molecular baskets. The β-cyclodextrins allow the loading and delivery of chemical cargoes.^{16,49} Synthesis of (β-CD was adapted from previously reported methods^{16,26,49} with some modifications. The CDs were diluted to 2 mg/L in a final volume of 10 mL using 10 mM TES buffer (pH 6.5). The CDs were sonicated for 30 min at 37 kHz and then filtered through a 20 nm filter (6809- 1002, Anotop, Whatman). Then, 0.5 mg of N-hydroxysulfosuccini-mide (NETS) and 0.2 mg 1-ethyl-3-(3-(dimethylamino)propyl) carbodiimide (EDC) were added to the solutions of CDs in 10 mM TES buffer (pH 6.5). The mixture was stirred for 30 min to activate carboxyl groups on the CDs. Following NHS/EDC activation, the solution 0.2 mg of 3-aminophenylboronic acid (APBA) was added dropwise to the reaction mixture and stirred at room temperature. Conjugation of APBA was allowed to react for 3 h at room temperature. The resulting APBA coated CDs were purified by washing three times with molecular biology grade water (catalog #46000CV Corning) through a 10 K Amicon filter (catalog #UFC901024, Amicon ultra, Merck Millipore). Then, the APBA coated CDs were sonicated for 30 min and 37 kHz and filtered through a 20 nm filter (6809-1002, Anotop, Whatman). The pH of the resulting solution was adjusted to 10.5 with NaOH in the 10 mM TES buffer. A 0.35 mg of mono-(6-ethanediamine-6-deoxy)-β-cyclodextrin (Cavcon) was added to the solution and allowed to react overnight at room temperature while stirring. The resulting β-cyclodextrin functionalized CDs were denoted β-CDs and purified by washing at least twice with a 10 K Amicon filter (catalog # UFC901024, Amicon ultra, Merck Millipore), then sonicated for 30 min at 37 kHz. The resulting β-CDs were filtered through a 20 nm filter (catalog # 6809-1002, Anotop, Whatman).

β-Cyclodextrin CD Functionalization with Targeting Peptide. Chloroplast targeting peptides were covalently bonded to β-CD by a stepwise conjugation.^{16,26} A double-ended cross-linker was used to attach β-CD to the targeting peptide. The succinimidyl-[(N-maleimidopropionamido)-tetraethylene glycol]ester (NHS-PEG4-MAL) (Thermo Fisher Scientific, USA) cross-linker contains chemical groups that are reactive to distinct functional groups located on the cyclodextrin molecule of the β-CD (terminal amine) and the targeting peptide's cysteine residue (sulfylhydrals). The 0.75 mg of NHS-PEG4-MAL linker was added to a solution of β-CD in a 10 mM TES buffer (pH 7.5). The mixture was incubated at room temperature for 1 h with stirring at 500 rpm. The excess NHS-PEG₄-MAL was removed by washing the mixture through a 10 K Amicon filter (catalog # UFC901024, Amicon ultra, Merch Millipore) using molecular biology grade water (catalog # 46000CV Corning), and the product was suspended in a 10 mM TES buffer (pH 7.0). Lastly, 0.75 mg of the RbcS chloroplast targeting peptide (MASSMLSSATMVGGC (SEQ ID NO:5)) was added to NHS-PEG₄-MAL activated β-CDs and allowed to react for 1 h at room temperature while stirring. The RbcS peptide was dissolved in a 1 mL solution of 0.1% DMSO and 10 mM TES buffer (pH 7.0). The resulting chloroplast targeting CD (TP-β-CD) was washed three times using a 10 K Amicon filter (catalog # UFC901024, Amicon ultra, Merch Millipore) with molecular biology-grade water (catalog # 46000CV Corning).

Chemical Cargo Loading in β-Cyclodextrin Molecular Baskets. To show proof of the concept of targeted chemical delivery by TP-β-CD, we utilized a model fluorescent dye, 6-carboxyfluorescein (FDA), that can form inclusion complexes with β-cyclo-dextrins.¹⁰⁵⁻¹⁰⁹ The FDA fluorescent dye has been reported to bind to the inner cavity of β-cyclodextrins and cyclodextrin derivatives for the investigation of chemical delivery by these molecular baskets in nonplant organisms.^{34,110} The loading of FDA fluorescent cargoes onto β-CD nanomaterials was carried out by adding approximately 0.4 mg of FDA to an aqueous solution of 20 mg/L TP-CD (0.2 mg) in 10 mM TES buffer (pH 7.0). The mixture was vortexed and incubated for 0.5 h and washed to remove unbound molecules through a 10 K Amicon filter (catalog # UFC901024, Amicon ultra, Merch Millipore) in 10 mM TES buffer (pH 7.0).

Characterization of Nanomaterials. All nanomaterials were characterized by absorbance UV-vis spectroscopy (UV-2600 Shimadzu), hydrodynamic size (Nano S), and ζ potential (Nano ZS). The nanomaterial ζ potential and hydrodynamic diameter were measured in a 10 mM TES buffer (pH 7.0). The ζ potential measurements were performed in 0.1 mM NaCl to improve conductivity and analyzed by the Hückel approximation model.¹¹¹ CD fluorescence emission was collected using a fluorescence spectrometer (Horiba PTI QM-400). The stepwise synthesis of TP-β-CDs was analyzed using FTIR (Thermo Nicolet 6700 FTIR).

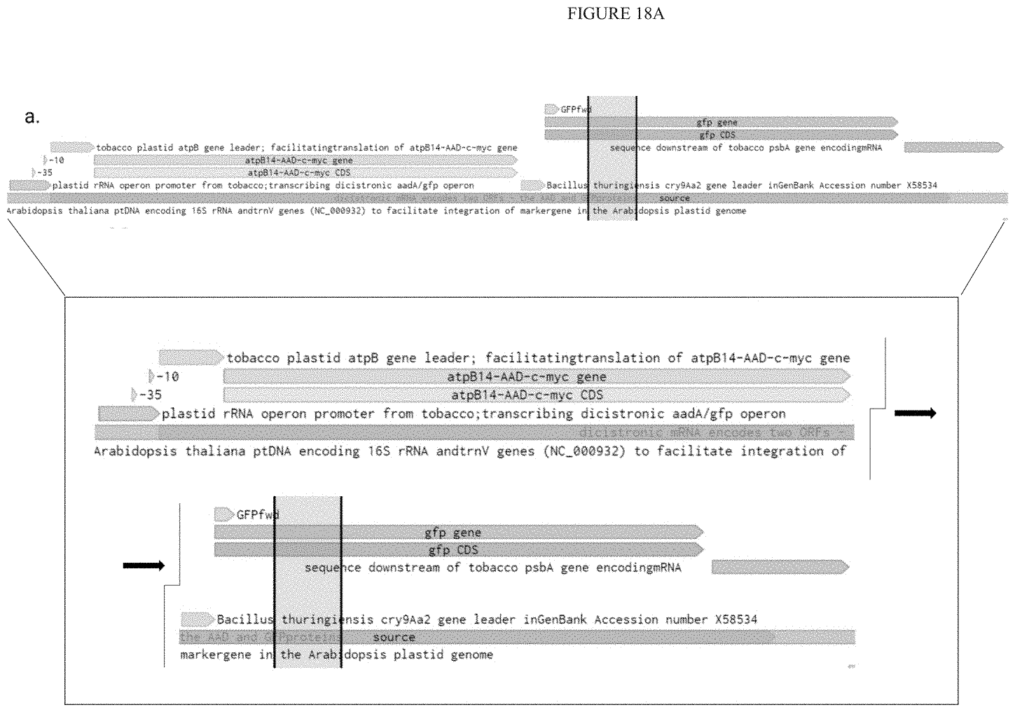

Nanomaterial Formulation and Topical Foliar Application. Nanomaterials were suspended in a 10 mM TES buffer (pH 7.0) with Silwet L-77 (Bio World) at a concentration used in agrochemical applications (0.1% v/v). The Silwet L-77 surfactant can reduce surface tension allowing rapid uptake into leaf stomatal pores and increase permeability in the epidermal layer through partial removal of the cuticular layer.¹⁸ Each formulation of nanomaterials was loaded into a 5 mL spray bottle allowing foliar application to the whole plant. Approximately 0.3 mL of the solution was dispensed with each foliar application. For all experiments, each plant replicate (5-12 plants) was treated with three foliar sprays of nanomaterials suspended in the formulation (Table 1) and allowed to interact with leaves for 1, 5, or 7 days. The amount of nanomaterials sprayed on plant leaves was estimated by weighing Arabidopsis plants on Petri dishes before and after foliar application of the formulation (FIG. 17). This experiment indicated that 34.2% of the total volume sprayed (900 μL) remains on the plant leaves. Thus, the amount of nanomaterials applied to plant leaves is β-CD (6.2 μg), TP-β-CD (6.2 μg), pATV1- SWCNT (0.62 μg), and TP-pATV1-SWCNT (0.62 μg).

Confocal Fluorescence Microscopy. Arabidopsis leaf samples were imaged by laser scanning confocal microscopy (TCS SP5, Leica Microsystems, Germany) using an ×40 wet objective (Leica Microsystems, Germany). Samples were dissected and mounted on glass slides inside a premade well of observation gel (Carolina). Confocal imaging of CDs (CD, β-CD, and TP-β-CD) and chloroplast autofluorescence were performed under 405 nm laser excitation (15% power) with an emission detection range set to 500-520 nm and 720-780 nm, respectively. The focal plane depth was set by adjusting the pinhole size to 3 airy units. To visualize the localization of chemical cargoes delivered by targeted CD complexes, a fluorescent 6-carboxyfluorescein dye (FDA, Invitrogen, catalog # C1360) was loaded into β-CDs and TP-β-CDs. The loading concentration of FDA to CDs was 2:1 as reported previously for β-cyclodextrins.^{34,105,109} The fluorescein dye was excited separately by a 488 nm laser at 40% power with a PMT emission detection range of 525-550 nm, and the pinhole size was set to 3 airy units. For confocal analysis of TP-CY3- ds(GT)₁₅-SWCNTs and CY3-ds(GT)₁₅-SWCNTs, the CY3 dye covalently linked to the DNA oligo (CY3-GTGTGTGTGTGTGTGTGTGTGTGTGTGTGT (SEQ ID NO:6)). The double-stranded oligo containing CY3 fluorophore was used to image the DNA-SWCNT in plant cells and chloroplasts. The CY3 labeled DNA oligo (CY3-GTGTGTGTGTGTGTGTGTGTGTGTGTGTGT(SEQ ID NO:6)) was purchased from IDT and annealed to a complementary DNA strand using the following procedure. An equal molar solution of each single stranded oligo was added to a tube containing 100 mM potassium acetate, 30 mM HEPES, pH 7.5 TBE buffer. The equal molar mixture was incubated at 95° C. for 5 min and allowed to cool expression analysis, images of 3-week-old Arabidopsis thaliana plants exposed to targeted nanomaterials after 1 and 5 days incubation, and list of primer sequences used for RT-qPCR. Methods for plant cell viability assays, chloroplast isolation, intact chloroplast analysis, leaf by a 543 nm laser (40% power) and photomultiplier tube (PMT) emission detection range set to 550-590 nm and the focal plane pinhole size to 3 airy units. For imaging GFP in plant leaves, confocal microscopy settings were 488 nm laser excitation and 500-530 nm fluorescence emission detection. The focal plane pinhole size was set to 3 airy units.

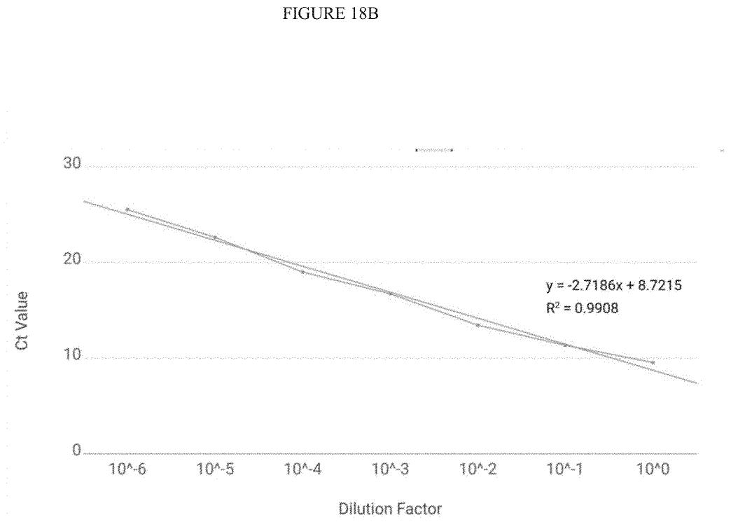

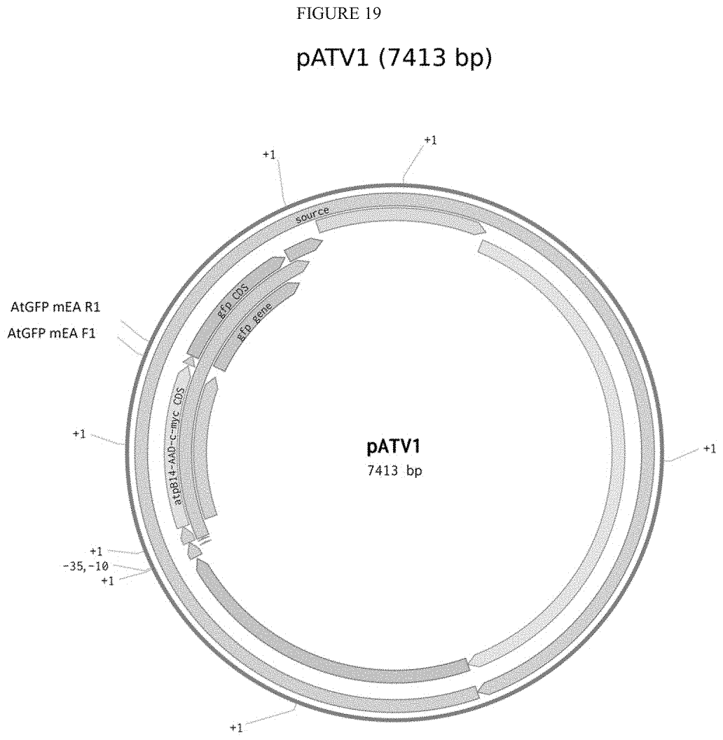

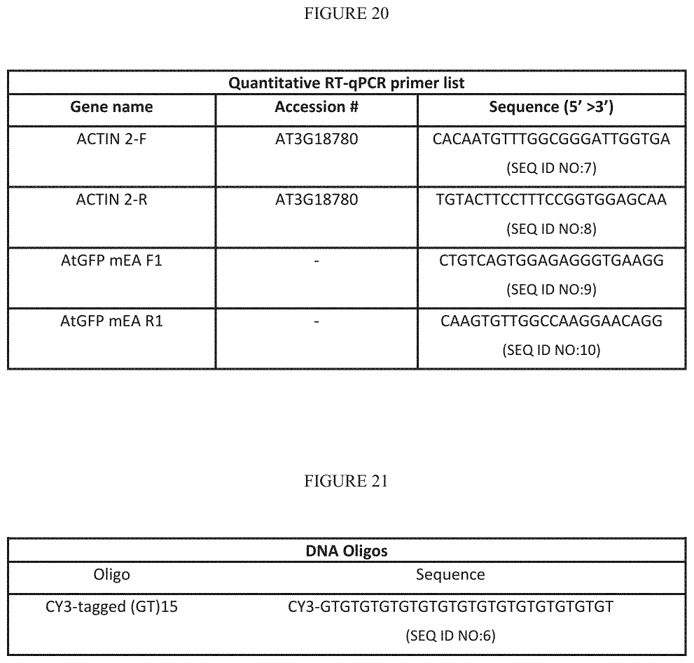

Real-Time Quantitative PCR Analysis. Expression analysis was performed on 3-week-old Arabidopsis leaves treated with 2 mg/L pATV1-SWCNTs, TP-pATV1-SWCNTs, and controls without nano-particles in 10 mM TES buffer (pH 7.0). Leaf RNA was extracted after 3 h of incubation with nanomaterials, 1, 5, and 7 days after treatment. The RNA was isolated using the Quick-RNA Plant Miniprep Kit (ZYMO). To digest any residual plasmid DNA carried over from into RT-qPCR reaction, samples were treated twice with DNase I enzyme (Zymo), while on the RNA plant miniprep column prep and after RNA was isolated. A 25 ng of purified RNA was added to Luna Universal One-Step RT-qPCR (NEB) reaction master mix per manufacturer's instructions. A quantitative real-time RT-qPCR was performed on a Bio-Rad CFX Connect Real-Time ThermalCycler (Bio-Rad). The relative expression levels of GFP genes were analyzed by the 2^{-ΔΔCT} method.⁶³ The gene Actin2 (AT3G18780) was used as internal housekeeping control. Expression analysis primers were designed with the Primer3 version 4.1.0 tool using pATV1 sequence as a template (FIG. 18A).^{43,104,112} The primers sets (FIG. 20) were validated for assessing gene expression (FIG. 18B).

Quantification of GFP by Enzyme-Linked Immunosorbent Assay. Leaf tissues treated with targeted TP-pATV1-SWCNTs and nontargeted pATV1-SWCNTs were ground under liquid nitrogen to a fine powder and homogenized in protein extraction buffer (50 mm Tris-HCl, pH 7.5, 150 mm NaCl, 0.1% [v/v] Triton X-100 and protease inhibitor cocktail). Total soluble proteins (TSP) were separated by centrifugation (12,000 g) for 15 min. The amount of GFP protein in TSP was measured using the GFP enzyme-linked immunosorbent assay kit (Cell Biolabs, San Diego, Calif., USA) according to the protocol provided by the manufacturer.

Statistical Analysis. We employed multiple strategies for statistical analysis depending on the number of variables and independent groups and whether comparisons were done for treatments with controls or across all treatments. Independent sample t tests were used to compare the means of two independent groups (i.e., FIGS. 3C and 5D). One-way ANOVA was performed to compare means of one variable across three or more independent groups (i.e., FIGS. 4C, 7B,D, 8, and 9A,B). Two-way ANOVA was performed to compare means of multiple treatments across several days (i.e., FIGS. 5E and 9C-F). Tukey's post hoc tests were used to compare every mean with every other mean (i.e., FIG. 4C). Dunnet's post hoc test was used to compare every mean to a control mean (i.e., FIGS. 6 and 7B,D).

TABLE 1
Summary of carbon nanostructures, plant material,
and nanomaterial concentration used in Example 1.

| FIG. | Nanomaterials | Plant material | Concentration |
|---|---|---|---|
| FIG. 3. Targeted | Targeted (TP-β-CD) and | Arabidopsis leaves | CD (20 mg/L) |
| delivery of | non-targeted carbon dots | | ds(GT)₁₅- |
| nanomaterials to | (β-CD) | | SWCNT |
| chloroplasts in plant | Targeted TP-CY3- | | (2 mg/L) |
| cells. | ds(GT)₁₅-SWCNT | | |
| | And non-targeted CY3- | | |
| | ds(GT)₁₅-SWCNT | | |
| FIG. 4. Chemical | Targeted TP-β-CD- | Arabidopsis leaves | 20 mg/L |
| cargo delivery by | FDA compared to non- | | |
| targeted carbon dot | targeted β-CD-FDA, | | |
| nanostructures. | FDA alone or a mixture | | |
| | of CD and FDA | | |
| FIG. 5. Plasmid | Targeted (TP- | Arabidopsis leaves | 2 mg/L |
| DNA delivery to | pATV1- SWCNT) | | |
| chloroplasts by | and non- targeted | | |
| targeted single-walled | SWCNT (pATV1- | | |
| carbon nanotubes | SWCNT) | | |
| FIG. 6. | Targeted carbon dots | Arabidopsis leaves | TP-β-CD (20, 100, |
| Biocompatibility of | (TP-β-CD) and | | 500 mg/L) |
| targeted | SWCNT (TP-pATV1- | | TP-pATV1- |
| nanomaterials in plant | SWCNT) | | SWCNT (2, 5, 10 |
| cells | | | mg/L) |
| FIG. 7. Impact of | TP-β-CD and TP- | Arabidopsis leaves | TP-β-CD (20 |
| targeted nanomaterials | pATV1-SWCNT | Isolated | mg/L) |
| on plant cell and | | chloroplasts | TP-pATV1- |
| chloroplast membrane | | | SWCNT (2 |
| integrity. | | | mg/L) |
| FIG. 8. Oxidative | TP-β-CD and TP- | Arabidopsis leaves | TP-β-CD (20 |
| stress in leaf | pATV1-SWCNT | Isolated | mg/L) |
| mesophyll cells | | chloroplasts | TP-pATV1- |
| exposed to targeted | | | SWCNT (2 |
| nanomaterials | | | mg/L) |
| FIG. 9. Effect of | TP-β-CD and TP- | Arabidopsis leaves | TP-β-CD (20 |
| targeted carbon | pATV1-SWCNT | | mg/L) |
| nanostructures on | | | TP-pATV1- |
| plant photosynthesis | | | SWCNT (2 |
| | | | mg/L) |

### Supporting Methods

### Plant Cell Viability Assays

The percentage of intact cells was determined in Arabidopsis thaliana leaf tissues treated with TP-β-CD (20, 100, 500 mg/L) or TP-pATV1-SWCNT (2, 5, 10 mg/L) from one to five days. The nuclei of dead cells were stained with propidium iodide (PI) diluted to 1× concentration manufactures (Plant cell viability assay kit, PA0100, Sigma-Aldrich). Leaf discs were collected and incubated in PI dye for 15 minutes before confocal analysis. PI can enter cells with damaged membranes, and bind to double-stranded nucleic acids in non-viable cells. The percentage of PI stained nuclei exhibited by dead cells versus total number of cells was determined through confocal microscopy. The total number of cells was determined using chloroplast autofluorescence to identify the leaf mesophyll cell boundaries. The leaf disc surface was washed with DI water to remove the residual dye and mounted on a microscope glass slide for imaging by laser scanning confocal microscope (TCS SP5, Leica Microsystems, Germany). Confocal imaging settings were as follows: ×40 wet objective (Leica Microsystems, Germany), propidium iodide dye excitation by a 543 nm laser (40% power), and PMT emission detection range set to 590-630 nm with Pinhole size set to 3 airy units.

### Chloroplast Isolation

Chloroplasts were isolated from Arabidopsis leaves through a centrifugation gradient method in sucrose buffer.¹⁻³ Intact chloroplasts were isolated from leaves exposed to targeted and non-targeted CDs and pATV1-SWCNT, and buffer as control (10 mM TES pH 7.3) in 0.1% Silwet (v/v) for one day or five days. Following foliar spray with nanomaterials as explained above, 4 g of leaf tissue was collected from seven plants per treatment. Treated tissue was harvested, and ground in ice-cold 1× chilled sucrose buffer (pH 7.3, 28 mM Na₂HPO₄, 22 mM KH₂PO₄, 2.5 mM MgCl₂, 400 mM sucrose, and 10 mM KCl). Following maceration of the leaf tissue, the homogenate was condensed to a pellet by two cycles of centrifugation at 4000 RCF for 10 minutes in a sucrose buffer and the supernatant was discarded. Isolated chloroplast suspension was stored in a 1× sucrose buffer at 4° C. for subsequent experiments such as DNA isolation or analysis of intact chloroplast.

### Intact Chloroplast Analysis

Identification of intact chloroplast was performed as reported in previous studies^{4,5} with some modifications. Following chloroplast isolation as indicated above, a 100 μL sample of isolated chloroplast solution was placed on a glass slide for examination by differential interference contrast (DIC) microscopy. Intact chloroplasts exhibit a highly reflective appearance with a bright and continuous halo around the envelope whereas damaged chloroplasts have a discontinuous halo with a granular appearance. The average from ten images from each of the seven biological replicates was quantified for calculating the average percentage of intact chloroplasts.

### Leaf H₂O₂ Quantification Assay

The H₂O₂ levels of Arabidopsis leaves treated with targeted nanomaterials (TP-β-CD or TP-pATV1-SWCNT) after one day or five days were assessed by a quantitative peroxide assay kit (Pierce, Thermo Scientific, USA). The leaf discs were collected at one and five days post foliar application treatment of nanomaterials and control samples. Two leaf discs were harvested from each biological replicate using a 6 mm cork borer, weighed immediately (0.02 g), and placed in a chilled mortar containing liquid nitrogen. The leaf disks were ground into a fine powder, transferred to a 2 mL microcentrifuge containing 0.5 mL of molecular biology grade water (Catalog # 46000CV Corning), and centrifuged at 13,000 RPM for 1 min. The 20 μL of supernatant collected was added to a plate reader well containing 200 μL of quantitative peroxide assay working reagent (0.25 mM ferrous ammonium sulfate, 100 mM sorbitol, 125 μm xylenol in 25 mM H₂SO₄). Samples were incubated for 15 minutes at room temperature, followed by measuring absorbance at 595 nm using an Infinite MPlex plate reader (Tecan).

### DNA Extraction from Leaves and Isolated Chloroplasts

DNA was extracted from plant leaf tissue and prepared using a Quick-DNA Plant/Seed DNA Miniprep kit (Zymo) in leaf samples treated with nanomaterials from one to five days. Each DNA sample contained 150 mg of plant tissue or 1 mL of isolated chloroplast suspension collected as explained above. Samples were placed in liquid nitrogen-filled mortar and pestle, ground, and placed directly into a Zymo BashingBead Lysis Tube with 750 μL BashingBead Buffer. The mixture was homogenized on a mixer mill (Retsch MM 400) for 10 minutes at 28 Hz. The DNA extraction was performed according to the manufacturer's instructions (zymoresearch.com) and concentrations were measured using a Nanodrop ND-1000 Spectrophotometer. Samples were stored at −20° C. until used for the 8-OHdG DNA damage biomarker assay.

### 8-OHdG DNA Damage Biomarker Assay

We determined the amount of oxidative DNA damage in plant leaves treated with nanomaterials for one day or five days using an enzyme-linked immunosorbent assay (ELISA) for 8-hydroxydeoxyguanosine (8-OHdG). The OxiSelect™ Oxidative DNA Damage ELISA Kit (Cat# STA-320, Cell Biolabs, inc.) quantifies the amount of 8-OHdG in isolated DNA samples from plant leaf tissue or isolated chloroplast suspensions. Briefly, isolated DNA plant material was converted from double to single-stranded during incubation at 95° C. for 5 minutes, followed by immediately placing it on ice. Single-stranded DNA was digested with the P1 nuclease enzyme (NEB) in 20 mM sodium acetate to convert it into single nucleotides. The digested DNA was treated with alkaline phosphatase (NEB) in 100 mM Tris (pH 7.5) and incubated for 1 hour at 37° C. to convert nucleotides into nucleosides for detection by antigen-specific 8-OHdG antibodies. The reaction mixture was centrifuged at 6,000×g for 5 minutes. The supernatant was collected for subsequent reaction with the 8-OHdG ELISA assay. The DNA Damage ELISA assay was conducted according to the manufacturer's instructions (Cell Biolabs, inc,). Sample absorbance was read at 450 nm on an Infinite MPlex plate reader (Tecan).

### Chlorophyll Measurements

Chlorophyll content index (CCI) was measured in plants treated one and five days after treatment with nanomaterials and buffer control solution (10 mM TES, pH 7.0). Three-week-old Arabidopsis plants were exposed to β-CD, TP-β-CD, pATV1-SWCNT, or TP-pATV1-SWCNT. The CCI measurements were performed using chlorophyll meters (SPAD-502 plus, Konica Minolta, Tokyo, Japan; CCI readout resolution: 0.1) with each leaf being measured three times for CCI readouts. The onset of leaf senescence was determined by a marked decrease in chlorophyll levels (FIG. 14B). Experiments before 4-weeks of development are ideal to avoid plant senescence symptoms such as the increase in programmed cell death rates and decrease in chlorophyll content.⁶⁻⁹

### Photosynthesis Assays

The photosynthetic capacity of Arabidopsis plants was performed using an infrared gas exchange analyzer (GFS-3000, Walz). Leaves from 3-week-old plants were exposed to TP-β-CD and TP-pATV1-SWCNTs or buffer as a control and measurements performed after one and five days. Leaves were placed inside a gas analyzer chamber ensuring that the leaf lamina was fully expanded to fill the entire measuring gas chamber 5 cm² (2.5 cm×1 cm). The CO₂ assimilation rates and PSII yield light response curves were performed at 1200, 900, 600, 400, 300, 200, 100, 50, and 0 PAR (μmol m⁻² s⁻¹). Leaf chamber settings were as follows: relative humidity 50%, CO₂ level 410 ppm, cuvette temperature 25° C., measurement time interval 210 seconds and flow rate 750 μmol/s. The Fᵥ/Fₘ dark-adapted measurements were performed after a 600-second dark interval under the leaf chamber conditions explained above.

Documents from the Supporting Methods of Example 1

(1) Weise, et al., Planta 2004, 218 (3), 474-482.

(2) Santana, et al., Nat. Commun. 2020, 11(1), 2045.

(3) Santana, et al., Bio Protoc 2021, 11 (12), e4060.

(4) Lilley, et al., New Phytol. 1975, 75 (1), 1-10.

(5) Kubis, et al., Methods Mol. Biol. 2008, 425, 171-186.

(6) Jung, S. Plant Physiol. Biochem. 2004, 42 (3), 225-231.

(7) Watanabe, et al., Plant Physiol. 2013, 162 (3), 1290-1310.

(8) Hortensteiner, et al., Biochim. Biophys. Acta 2011, 1807 (8), 977-988.

(9) Bieker, et al., J. Integr. Plant Biol. 2012, 54 (8), 540-554.

### Documents in Example 1

(1) Alexandratos, N.; Bruinsma, J. World Agriculture towards 2030/2050: The 2012 Revision. ESA Working Paper no. 12-03; Food and Agriculture Organization of the United Nations: Rome, 2012.

(2) Ray, et al., PLoS One 2013,8 (6), No. e66428.

(3) Kah, et al., Nat. Nanotechnol. 2019, 14(6), 532-540.

(4) United States EPA. Climate Impacts on Agriculture and Food Supply; United States Environmental Protection Agency: Washington, D.C., 2016.

(5) Schlenker, et al., Environ. Res. Lett. 2010,5 (1), 014010.

(6) Willett, et al., Lancet 2019,393 (10170), 447-492.

(7) Mba, et al., Agriculture & Food Security 2012, 1 (1), 7.

(8) Lowry, et al., Nat. Nanotechnol. 2019,14 (6), 517-522.

(9) Newkirk, et al., Advancements. Front. Plant Sci. 2021,12, 1496.

(10) Hofmann, et al., Nature Food 2020, 1 (7), 416-425.

(11) Wang, et al., Mol. Plant 2019,12 (8), 1037-1040.

(12) Giraldo, et al., Nat. Nanotechnol. 2019,14 (6), 541-553.

(13) Wang, et al., Nat. Nanotechnol. 2022, 17,347-360.

(14) Servin, et al., Nanampact 2016, 1, 9-12.

(15) Lv, et al., Environmental Science:Nano 2019,6 (1), 41-59.

(16) Santana, et al., Nat. Commun. 2020,11 (1), 2045.

(17) Wang, et al., Trends Plant Sci. 2016, 21 (8), 699-712.

(18) Hu, et al., ACS Nano 2020, 14 (7),7970-7986.

(19) Kwak, et al., Nat. Nanotechnol. 2019, 14 (5), 447-455.

(20) Avellan, et al., ACS Nano 2019, 13 (5), 5291-5305.

(21) Su, et al., Environ. Sci.: Nano 2019, 6 (8), 2311-2331.

(22) Lew, et al., Small 2018, 14 (44), No. 1802086.

(23) Wong et al., Nano Lett. 2016,16 (2), 1161-1172.

(24) Demirer, et al., Nat. Nanotechnol. 2019, 14, 456-464.

(25) Spielman-Sun, et al., Trichomes. Nanoscale 2020, 12, 3630-3636.

(26) Santana, et al., Bio Protoc 2021, 11 (12), No. e4060.

(27) Marmiroli, et al., Chemosphere 2020, 240, 124856.

(28) Majumdar, et al., Environmental Science: Nano 2019, 6 (10), 3010-3026.

(29) Li, et al., Materials Chemistry Frontiers 2020, 4 (2), 437-448.

(30) Li, et al., ACS Appl. Mater. Interfaces 2016, 8 (31), 19939-19945.

(31) Swift, et al., New Phytol. 2021, 229 (2), 783-790.

(32) Li, et al., Nano Res. 2019, 12, 1585-1593.

(33) Khan, et al., Sci. Rep. 2017, 7 (1), 14866.

(34) Zhu, et al., Talanta 2007, 72 (1), 237-242.

(35) Saha, et al., Sci. Rep. 2016, 6, 35764.

(36) Zhang, et al., Nat. Protoc. 2020, 15 (9), 3064-3087.

(37) Demirer, et al., Nat. Protoc. 2019, 14, 2954-2971.

(38) Jackson, et al., ACS Nano 2022, 16 (2), 1802-1812.

(39) Sanzari, et al., Frontiers in Bioengineering and Biotechnology 2019, 7, 120.

(40) Begum, et al., J. Hazard. Mater. 2012, 243, 212-222.

(41) Lin, et al., Small 2009, 5 (10), 1128-1132.

(42) Tripathi, et al., Plant Physiol. Biochem. 2017, 110, 2-12.

(43) Yu, et al., Plant Physiol. 2017, 175 (1), 186-193.

(44) Zhu, et al., Angew. Chem., Int. Ed. Engl. 2013, 52 (14), 3953-3957.

(45) Holá, et al., ACS Nano 2017, 11 (12), 12402-12410.

(46) Wang et al., Sci. Rep. 2019, 9 (1), 10723.

(47) Wen, et al., Arabian Journal of Chemistry 2017, 10, S1680—S1685.

(48) Mazumdar, et al., Nanomaterials 2020, 10 (2), 325.

(49) Tang, et al., Biosensors and Bioelectronics 2016, 83, 274-280.

(50) Li, et al., J. Hazard. Mater. 2021, 410, 124534.

(51) Mondal, et al., J. Phys. Chem. C 2016, 120 (26),14365-14371.

(52) Kranjc, et al., Environ. Sci.: Nano 2018, 5 (2), 520-532.

(53) Xia, et al., Explorationen 2021, 1 (1), 9-20.

(54) Demirer, et al., Nat. Nanotechnol. 2019,14 (5), 456-464.

(55) Hirayama, et al., Adv. Drug Delivery Rev. 1999, 36 (1), 125-141.

(56) Li, et al., J. Phys. Chem. C 2010, 114 (28), 12062-12068.

(57) Amer et al., J. Drug Delivery Sci. Technol. 2020, 55, 101408.

(58) Alas, et al., J. Mater. Sci. 2020, 55 (31), 15074-15105.

(59) Smith, et al., ACS Sustainable Chem. Eng. 2018, 6 (11), 13599-13610.

(60) Ng, et al., PLoS One 2016, 11 (4), No. e0154081.

(61) Yoshizumi, et al., Biomacromolecules 2018, 19 (5), 1582-1591.

(62) Chen, et al., Gene Ther. 2000, 7 (19), 1698-1705.

(63) Pfaffl, et al., Nucleic Acids Res. 2001, 29 (9), No. e45.

(64) Livak, et al., Methods 2001, 25 (4), 402-408.

(65) Liu, et al., Plant J. 2007, 51 (6), 941-954.

(66) Wang, et al., Curr. Opin. Plant Biol. 2018, 45,171-177.

(67) Lilley, et al., New Phytol. 1975, 75 (1), 1-10.

(68) Kubis, et al., Methods Mol. Biol. 2008, 425, 171-186.

(69) Asada, K. Plant Physiol. 2006, 141(2), 391-396.

(70) Veljovic-Jovanovic et al., Plant Physiol. Biochem. 2002, 40 (6), 501-507.

(71) Cheeseman, J. M. J. Exp. Bot. 2006, 57 (10), 2435-2444.

(72) C̆erný, et al., Int. J. Mol. Sci. 2018, 19 (9), 2812.

(73) Tripathi, et al., Front. Plant Sci. 2020,11, 596.

(74) Cheeseman, et al., Plant stress 2007, 1 (1), 4-15.

(75) Hung, et al., J. Plant Physiol. 2004, 161 (12), 1347-1357.

(76) Bieker, et al., J. Integr. Plant Biol. 2012, 54 (8), 540-554.

(77) Das, et al., Front. Environ. Sci. Eng. China 2014, 2, 53.

(78) Chi, et al., Front. Plant Sci. 2013, 4, 277.

(79) Valavanidis, et al., J. Environ. Sci. Health C Environ. Carcinog.Ecotoxicol. Rev. 2009, 27 (2), 120-139.

(80) Chiou, et al., Clin. Chim. Acta 2003, 334 (1), 87-94.

(81) Hu, et al., Mutat. Res. 1995, 336 (2), 193-201.

(82) Kumar, et al., J. Exp. Bot. 2014, 65 (22), 6425-6439.

(83) Oldenburg, et al., Front. Plant Sci. 2015, 6, 883.

(84) Aarti, et al., Physiol. Plant. 2006, 128 (1), 186-197.

(85) Mukherjee, et al., Environ. Monit. Assess. 2019, 191 (6), 400.

(86) Kalaji, et al., Acta Physiol. Plant 2016, 38 (4), 102.

(87) Giraldo, et al., Nat. Mater. 2014, 13 (4), 400-408.

(88) Walkey, et al., Chem. Soc. Rev. 2012, 41 (7), 2780-2799.

(89) Foroozandeh, et al., Nanoscale Res. Lett. 2015, 10, 221.

(90) Liu, et al., Nanoscale 2013, 5 (4), 1658-1668.

(91) Borgatta, et al., Environ. Sci.: Nano 2021, 8 (4), 1067-1080.

(92) Genty, et al., Biochimica et Biophysica Acta (BBA)—General Subjects 1989, 990 (1), 87-92.

(93) Maxwell, et al., J. Exp. Bot. 2000, 51 (345), 659-668.

(94) Jarvis, et al., Biochim. Biophys. Acta 2002, 1590 (1-3), 177-189.

(95) Lee, et al., Biochim. Biophys. Acta 2013, 1833 (2), 245-252.

(96) Nafees, M.; Fahad, S.; Shah, A. N.; Bukhari, M. A.; Maryam; Ahmed, I.; Ahmad, S.; Hussain, S. Reactive Oxygen Species Signaling in Plants. In Plant Abiotic Stress Tolerance: Agronomic, Molecular and Biotechnological Approaches; Hasanuzzaman, M.; Hakeem, K. R.; Nahar, K.; Alharby, H. F., Eds.; Springer International Publishing: Cham, 2019; pp 259-272.

(97) Watanabe, et al., J. Plant Res. 2006, 119 (3), 239-246.

(98) Claeys, et al., Plant Physiol. 2014, 165 (2), 519-527.

(99) Smirnoff, et al., New Phytol. 2019, 221 (3), 1197-1214.

(100) Ramos-Perez, et al., Methods Mol. Biol. 2013, 1025, 261-268.

(101) Lee, et al., Plant Physiol. 2009, 151 (1), 129-141.

(102) Lakshmanan, et al., Biomacromolecules 2013, 14 (1), 10-16.

(103) Chen, et al., Adv. Drug Delivery Rev. 2013, 65 (10), 1357-1369.

(104) Yu, et al., Nat. Plants 2019, 5 (5), 486-490.

(105) Flamigni, L. J. Phys. Chem. 1993, 97 (38), 9566-9572.

(106) Dong, et al., Small 2013, 9 (3), 446-456.

(107) Angelini, et al., Chem. Phys. Lipids 2017, 209, 61-65.

(108) Brittain, H. G. Chem. Phys. Lett. 1981, 83 (1), 161-164.

(109) Hamada, et al., J. Inclusion Phenom. Mol. Recognit. Chem. 1996, 25 (4), 283-294.

(110) Li, et al., RSC Adv. 2015, 5 (84), 68815-68821.

(111) Doane, et al., Acc. Chem. Res. 2012, 45 (3), 317-326.

(112) Koressaar, et al., Bioinformatics 2007, 23 (10), 1289-1291.

The entire content of Santana et al., ACS Nano 2022, 16, 8, 12156-12173 is incorporated by reference herein.

All publications, patents, and patent documents are incorporated by reference herein, as though individually incorporated by reference. The present disclosure has been described with reference to various specific and preferred embodiments and techniques. However, it should be understood that many variations and modifications may be made while remaining within the spirit and scope of the invention.

What is claimed is:

1. A polypeptide comprising a chloroplast-targeting amino acid sequence linked to an amino acid sequence that is capable of electrostatically binding to a nucleic acid molecule.

2. The polypeptide of claim 1, wherein the chloroplast-targeting amino acid sequence comprises an amino acid sequence of a ribulose biosphosphate carboxylase small chain 1A (RBCS1A) protein.

3. The polypeptide of claim 1, wherein the chloroplast-targeting amino acid sequence comprises an amino acid sequence having at least about 75%, 80% or 90% sequence identity to
(SEQ ID NO: 1)
MASSMLSSATMV.

4. The polypeptide of claim 3, wherein the chloroplast-targeting amino acid sequence comprises MASSMLSSATMV (SEQ ID NO:1).

5. The polypeptide of claim 1, wherein the amino acid sequence that is capable of electrostatically binding to a nucleic acid molecule is lysine- and histidine-rich.

6. The polypeptide of claim 5, wherein the amino acid sequence that is capable of electrostatically binding to a nucleic acid molecule comprises an amino acid sequence having at least about 75%, 80%, 85%, 90%, or 95% sequence identity to KHKHKHKHKHKH (SEQ ID NO:2).

7. The polypeptide of claim 5, wherein the amino acid sequence that is capable of electrostatically binding to a nucleic acid molecule comprises KHKHKHKHKHKH (SEQ ID NO:2).

8. The polypeptide of claim 1, wherein the chloroplast-targeting amino acid sequence is linked to the amino acid sequence that is capable of electrostatically binding to a nucleic acid molecule by a peptide bond or by a polypeptide linker.

9. The polypeptide of claim 8, wherein the chloroplast-targeting amino acid sequence is linked to the amino acid sequence that is capable of electrostatically binding to a nucleic acid by a polypeptide linker, wherein the polypeptide linker is GGGGGG (SEQ ID NO:3).

10. The polypeptide of claim 1, which comprises an amino acid sequence having at least about 75%, 80%, 85%, 90%, or 95% sequence identity to
(SEQ ID NO: 4)
MASSMLSSATMVGGGGGGKHKHKHKHKHKH.

11. The polypeptide of claim 10, which comprises
(SEQ ID NO: 4)
MASSMLSSATMVGGGGGGKHKHKHKHKHKH.

12. A polynucleotide comprising a nucleic acid sequence encoding the polypeptide of claim 1.

13. A conjugate comprising one or more polypeptides as described in claim 1 and a nucleic acid, wherein the one or more polypeptides are electrostatically bound to the nucleic acid.

14. A conjugate comprising one or more polypeptides as described in claim 1, one or more nucleic acids, and a functionalized nanoparticle, wherein the one or more nucleic acids are electrostatically bound to the functionalized nanoparticle, wherein the one or more polypeptides are electrostatically bound to the one or more nucleic acids, and wherein the functionalized nanoparticle is selected from the group consisting of a functionalized quantum dot, carbon dot, carbon nanotube, silica nanoparticle, and metal or metal oxide nanoparticle.

15. The conjugate of claim 14, wherein the nucleic acid is DNA.

16. The conjugate of claim 14, wherein the functionalized nanoparticle is coated with polyethyleneimine (PEI) and/or ethylenediamine (EDA), which comprise amines that are capable of being protonated.

17. The conjugate of claim 16, wherein the functionalized nanoparticle is coated with PEI, and wherein the functionalized nanoparticle is a single walled carbon nanotube (SWNT).

18. A composition comprising the polypeptide of claim 1 and a carrier.

19. A method of delivering one or more nucleic acids to a chloroplast of a plant, the method comprising introducing to the plant a conjugate as described in claim 14.

20. A method of expressing a target RNA or protein in the chloroplast of a plant, the method comprising introducing to the plant a conjugate as described in claim 14, wherein the conjugate comprises one or more nucleic acids encoding the target RNA or protein.

21. A method of treating a disease in a plant, the method comprising introducing to the plant an effective amount of a conjugate as described in claim 14, wherein the conjugate comprises one or more nucleic acids effective to treat the disease.
